(19) United States
(12) Patent Application Publication
(10) Pub. No.: US 2024/0085433 A1
(43) Pub. Date: Mar. 14, 2024

(54) METHODS OF USING MESOTHELIN

(71) Applicants: Amy Ryan, Toddville, IA (US); Noa Harriott, Iowa City, IA (US)

(72) Inventors: Amy Ryan, Toddville, IA (US); Noa Harriott, Iowa City, IA (US)

(21) Appl. No.: 18/458,619

(22) Filed: Aug. 30, 2023

Related U.S. Application Data
(60) Provisional application No. 63/402,145, filed on Aug. 30, 2022.

(51) Int. Cl. G01N 33/68; A61K 39/00; A61K 45/06; C07K 16/10; G01N 33/569

(52) U.S. Cl. CPC G01N 33/6893; A61K 39/4611; A61K 39/4621; A61K 39/4631; A61K 39/4643; A61K 45/06; C07K 16/1003; G01N 33/56983; A61K 2039/505; G01N 2800/56

(57) ABSTRACT

Methods of using mesothelin levels is provided.

### CROSS-REFERENCE TO RELATED APPLICATIONS

This application claims the benefit of the filing date of U.S. application No. 63/402,145, filed on Aug. 30, 2022, the disclosure of which is incorporated by reference herein.

### BACKGROUND

Beginning in 2019, the global coronavirus disease (COVID-19) pandemic has presented a large-scale public 46 health challenge, with the death toll in the United States exceeding 1 million (https://covid.edc.gov/covid-data-tracker/#datatracker-home), and the global death count over 6 million as of July 2022 (https://covid19.who.int/). COVID-19, which results from infection by the severe acute respiratory syndrome coronavirus 2 (SARS-CoV-2) virus (Zhu et at., 2020), is a disease primarily characterized by dry cough, fever, and fatigue. However, symptoms can also include sore throat, shortness of breath, loss of smell and/or taste, headache, chills, nausea, vomiting, and diarrhea (Guan et al., 2020; Song et al., 2021; Wang et al., 2020). Symptoms can also persist long after resolution of the initial infection, in some cases more than 14 months (Rays et al., 2022; de Miranda et al., 2022; Desai et al., 2022). In addition to a wide variety of symptoms, COVID-19 is associated with significant variation in disease severity. While the majority of cases are mild or asymptomatic (>85%), about 14% of cases require hospitalization, and about 2% of all cases are lethal (Wu & McGoogan, 2020).

Clinical observations rapidly identified age as a primary risk factor for hospitalization and mortality (Schafer et al., 2022; Buttenschon et al., 2022). Age-related risk for severe COVID-19 has been a core focus of scientific investigation, and a variety of plausible explanations have been presented in the literature. Expression of angiotensin converting enzyme 2 (AGE2), the primary cell surface receptor for SARS-CoV-2 (Letko et al., 2020; Hoffmann et al., 2020), in addition to other SARS-CoV-2 entry factors, has been proposed as an explanation for age-related differences, with higher expression levels of these entry factors being detected in the nasal epithelium of older patients (Singh et al., 2020; Abrehart et al., 2022). In addition to the airway epithelium, ACE2 is also expressed on endothelial cells (Ferrario et al., 2005), where infection of the endothelial lining has been proposed to contribute to endotheliitis observed in severe COVID-19 (Varga et al., 2020). In general, children have comparatively healthier endothelium, potentially contributing to age-related differences in COVID-19 severity (Zimmermann & Curtis. 2022). Other studies, however, have found no correlation between proportion of ACE2+ cells and disease severity (Ma et al., 2021).

Building on these hypotheses, a more robust innate immune response in response to infection with SARS-CoV-2 is observed in children compared to adults (Loske et al., 2022; Pierce et al., 2021), and a dysregulation of innate immune function, similar to the “inflammaging” reported in aged populations, is observed in severe COVID-19 (Schultze & Aschenbrenner, 2021). Increased efficiency of the adaptive immune system is associated with children, and while adults may mount a more activated response, children are known to maintain higher levels of regulatory cells and memory T-cells (Loske et al., 2022; Petrara et al., 2021; Cohen et al., 2021). Increased activation of the adaptive immune system in this context appears to be detrimental and results in increased T-cell exhaustion. In this case, chronic T-cell activation results in the impaired function commonly observed in older patients, and correlates to COVID-19 disease severity (Blank et al., 2019; Palmer et al., 2021; Yang et al., 2020; Westmeier et al., 2020). This aberrant regulation of the immune response can also be maintained over time (Files et al., 2021), a finding with implications for the investigation of “long haul COVID”.

### SUMMARY

Plasma samples from a COVID-19 biorepository, which were obtained from patients with mild, moderate, and severe COVID-19, were analyzed using protein analysis. Specifically, using O-Links protein expression analysis platform, certain proteins such as mesothelin (MSLN) were found to be significantly higher in concentration in the plasma of severe COVID-19 patients and concentrations were reduced with severity. As MSLN was detected in blood plasma in soluble form, MSLN may be a biomarker for severe COVID-19 and may underlie new blood vessel formation in the lung contributing to increased blood flow complications and potentially long-haul COVID. MSLN may also be a therapeutic target for intervention in COVID-19 and potentially long-haul COVID-19. For example, antibodies to MSLN or CAR-T therapy against MSLN may be employed in patients infected with SARS-CoV-2.

In one embodiment, the disclosure provides a method to determine an increased risk of developing severe COVID in a mammal, comprising: determining an amount of mesothelin in a physiological sample, e.g., plasma or serum, from a test mammal infected or suspected of being infected with SARS-CoV-2; and determining whether the amount of mesothelin in the test mammal is greater than in a corresponding sample from a corresponding mammal that does not have severe COVID or is similar to amounts in a corresponding sample in a corresponding mammal with severe COVID, wherein if the amount in the test mammal is greater than the mammal that does not have severe COVID or is similar to the mammal with severe COVID, the test mammal is at increased tisk of developing severe COVED. Amounts of MSLN in a physiological fluid sample that are associated with progression to severe COVID-19 disease and occurrence of long-haul syndrome include but are not limited to at least at least 30 ng/mL, at least 35 ng/mL, at least 40 ng/mL, at least 50 ng/mL or greater. Thus, a “similar amount” in a physiological fluid sample is about 30 ng/mL, to about 70 ng/ml. Amounts of MSLN in a physiological fluid sample that are less than about 30 ng/mL, less than about 25 ng/mL, less than about 20 ng/mL or less than about 15 ng/mL or less are associated with moderate COVID, mild COVID, or an uninfected mammal. In one embodiment, the mammal that does not have severe COVID has moderate COVID. In one embodiment, the mammal that does not have severe COVID has mild COVID. In one embodiment, the mammal that does not have severe COVID does not have COVID. In one embodiment, the mammal is a human. In one embodiment, the method further comprises treating the test mammal having greater amounts of mesothelin in the plasma or serum with one or more compounds. In one embodiment, the one or more compounds include an anti-viral compound or an anti-mesothelin mesothelin compound, or a combination thereof. In one embodiment, the one or more anti-viral compounds include an antibody or fragment thereof. In one embodiment, the antibody is a monoclonal antibody or a fragment thereof. In one embodiment, the one or more anti-viral compounds include a protease inhibitor. In one embodiment, the one or more anti-viral compounds include a ribonucleoside. In one embodiment, the one or more compounds include a steroid. In one embodiment, the steroid comprises dexamethasone. In one embodiment, the one or more compounds include ritonavir, paxlovid (nirmatrelvir), molnupiravir, remdesivir, bebtelovimab, or ivermectin. In one embodiment, the one or more anti-mesothelin compounds comprise CAR-T therapy. In one embodiment, the one or more compounds include an IL-6 inhibitor such as tocilzumab or sarilumab. In one embodiment, the one or more compounds include a JAK inhibitor such as baricitinib or tofacitinib.

In one embodiment, the disclosure provides a method to prevent, inhibit or treat SARS-CoV-2 infection in a mammal, comprising administering an effective amount of one or more compounds to a mammal determined to have increased mesothelin levels in a physiological sample, e.g., plasma or serum, relative to a corresponding sample in a corresponding mammal that is infected with SARS-CoV-2 and does not have severe COVID or a corresponding sample in a corresponding mammal that is not infected with SARS-CoV-2 or determined to have similar levels of mesothelin relative to a corresponding sample in a corresponding mammal with severe COVID. In one embodiment, the corresponding mammal has moderate COVID. In one embodiment, the corresponding mammal has mild COVID. In one embodiment, the mammal is a human. In one embodiment, the one or more compounds include an anti-viral compound or an anti-mesothelin compound, or a combination thereof. In one embodiment, the one or more anti-viral compounds include an antibody or fragment thereof. In one embodiment, the antibody is a monoclonal antibody or a fragment thereof. In one embodiment, the one or more anti-viral compounds include a protease inhibitor. In one embodiment, the one or more anti-viral compounds include a ribonucleoside. In one embodiment, the one or more compounds include a steroid. In one embodiment, the one or more anti-mesothelin compounds comprise CAR-T therapy. In one embodiment, the one or more compounds include an IL-6 inhibitor such as tocilzumab or sarilumab. In one embodiment, the one or more compounds include a JAK inhibitor such as baricitinib or tofacitinib.

In one embodiment, a method to determine risk of developing long COVID in a mammal is provided comprising: determining an amount of mesothelin in plasma from a mammal having COVID, e.g., within 5 days to 2 months after infection; and determining whether the amount of mesothelin in a physiological sample, e.g., plasma or serum, in the mammal, e.g., over time, is greater than in a corresponding sample from a corresponding mammal that does not have severe COVID or similar amounts to that in a corresponding sample from a corresponding mammal with severe COVID, wherein if the amount in the mammal having COVID is greater than the corresponding mammal that does not have severe COVID or similar to the mammal with severed COVID, the mammal is at an increased risk of developing long COVID.

In one embodiment, a method to determine whether a patient with COVID is a candidate for admission to an intensive care unit is provided comprising: determining an amount of mesothelin in a physiological sample, e.g., plasma or serum, from the patient; and determining whether the amount of mesothelin in the patient is greater than a corresponding sample from a patient that does not have severe COVID or similar to that in a corresponding sample from a patient with severe COVID, wherein if the amount in the patient with COVID is greater than the patient that does not have severe COVID or similar to a patient with severe COVID, the patient is a candidate for admission to the ICU.

In one embodiment, a screening method is provided comprising; combining human mesothelin and one or more compounds; and determining whether the one or more compounds bind to human mesothelin or inhibit the activity of human mesothelin.

In one embodiment, a method to prevent, inhibit or treat SARS-CoV-2 infection in a mammal is provided, comprising administering to a mammal having increased mesothelin levels in a physiological sample, e.g., plasma or serum, relative to a corresponding sample from a corresponding mammal that is infected with SARS-CoV-2 and does not have severe COVID or has similar amounts of mesothelin to that is a corresponding sample from a corresponding mammal with severe COVID, an amount of one or more compounds that bind to or inhibit mesothelin.

### BRIEF DESCRIPTION OF THE FIGURES

FIGS. 1A-1B. Plasma protein expression signatures are associated with severity of COVID-19. FIG. 1A: Principal component analysis (PCA) plot comparing samples across all cohorts. FIG. 1B: Heatmap of relative protein expression. Proteins were evaluated through an unbiased hierarchical clustering according to scaled NPX, value for each sample.

FIGS. 2A-2B. Network analysis identifies markers of inflammation and cell proliferation in severe COVID-19. FIG. 2A: STRING network analysis map of all significantly differentially expressed proteins (DEPs) in severe vs. any other cohort. Lines indicate known associations between proteins. Thickness of line indicates confidence score (minimum=0.4). FIG. 2B: Top 10 most enriched pathways as determined by Ingenuity Pathway Analysis (IPA) of all significant DEPs in severe vs any other cohort.

FIGS. 3A-3J. Markers of inflammation and cell proliferation are expressed significantly higher in severe COVID-19. FIGS. 3A-3C: Volcano plots showing DEPs between severe and mild (FIG. 3A), severe and moderate (FIG. 3B), and severe and control cohorts (FIG. 3C). Dotted line indicates an unadjusted p-value=0.05; significance was determined by adjusted p-value<0.05. FIG. 3D: Venn diagram overlaying DEPs between severe and mild (green), severe and moderate (orange), and severe and control cohorts (blue). FIGS. 3E-3G: Cluster plots of normalized protein expression (NPX) values by cohort. Syndecan-1 (FIG. 3E), EN-RAGE (FIG. 3F), and Mesothelin (FIG. 3G). FIGS. 3H-3J: Cluster plots of NPX values in severe and moderate cohorts over collection days 1, 3, and 5. Mild values at day 1 included for reference. Interleukin-18 Receptor 1 (FIG. 3H), Hepatocyte Growth Factor (FIG. 3I), and CXCL10 (FIG. 3J). For all cluster plots data is presented as: *p0.05, **p<0.01, ***p<0.001, ****p<0.0001. Black horizontal line indicates the median.

FIGS. 4A-4J. A subset of “protective” proteins are significantly higher in mild disease also correlate to younger age groups. FIGS. 4A-4C: Volcano plots showing DEPs between mild and severe (FIG. 4A), mild and moderate (FIG. 4B), and mild and control cohorts (FIG. 4C). Dotted line indicates an unadjusted p-value of 0.05; significance was determined by adjusted p-value<0.05. FIG. 4D: Venn diagram overlaying DEPs between mild and severe (green), mild and moderate (orange), and mild and control cohorts (blue). FIGS. 4E-4H: Cluster plots of normalized protein expression (NPX) values by cohort. TRANCE (FIG. 4E), Fas Ligand (FIG. 4F), XPNPEP2 (FIG. 4G), and CD207 (FIG. 4H). FIGS. 4I-4J: Cluster plots of NPX values by age group for TRANCE (FIG. 4I) and Fas Ligand (FIG. 4J). For all cluster plots: *p<0.05, **p<0.01, ***p<0.001, ****p<0.0001. Black horizontal line indicates the median.

FIGS. 5A-5B. Cluster plot of normalized protein expression (NPX) values by cohort (FIG. 5A) and summary of disease status and candidate markers (FIG. 5B).

FIG. 6. Osteoprotegerin (OPG) expression shown by COVID-19 severity cohort. Cluster plot of normalized protein expression (NPX) values by cohort for severe (N=21), moderate (N=21), mild (N=10), and control (N=16) cohorts. All data points are shown. **p<0.01, ****p<0.0001.

FIG. 7. COVID-19 severe disease sample. Increased airway epithelial expression of Mesothelin.

FIG. 8. Non-COVID-19 deceased donor sample.

FIG. 9. Non-COVID-19 lung tissue. KRT5 (green) airway basal cells SMA (cyan) smooth muscle and myoepithelial cells of the sub mucosal glands. MESO (red) Mesothelin-expressing cells. DAPI-Nuclei

FIG. 10. End-stage COVID-19 tissue (Severe), KRT5 (green) airway basal cells. SMA (cyan) smooth muscle and myoepithelial cells of the sub mucosal glands. MESO (red) Mesothelin-expressing cells. DAPI-Nuclei.

FIG. 11. End-stage COVID-19 tissue (Severe). KRT5 (green) airway basal cells. SMA (cyan) smooth muscle and myoepithelial cells of the sub mucosal glands. MESO (red) Mesothelin-expressing cells. DAPI-Nuclei.

FIG. 12. End-stage COVID-19 tissue (Severe). KRT5 (green) airway basal cells. SMA (cyan) smooth muscle and myoepithelial cells of the sub mucosal glands. MESO (red) Mesothelin-expressing cells. DAPI-Nuclei.

FIGS. 13A-13B. Plots of MSLN (FIG. 13A) and MUC16 (FIG. 13B) in non-COVID19 and severe COVID19 samples.

FIGS. 14A-14B. Cell specific expression of MSLN (FIG. 14A) and MUC16 14B).

### DETAILED DESCRIPTION

SARS-CoV-2 infection remains a major public health concern, particularly for the aged and those individuals with co-morbidities at risk for developing severe COVID-19. Understanding the pathogenesis and biomarkers associated with responses to SARS-CoV-2 infection remain components in developing effective therapeutic approaches, especially in cases of severe and long-COVID-19. In the study herein, blood plasma protein expression was compared in subjects with mild, moderate, and severe COVID-19 disease. Evaluation of an inflammatory protein panel confirmed upregulation of proteins including TNFβ, IL-6, IL-8, and IL-12, already associated with severe cytokine storm and progression to severe COVID-19. Several proteins not yet associated with COVID-19 disease, including mesothelia (MSLN), that are expressed at significantly higher levels in severe COVID-19 subjects were identified. In addition, a subset of markers associated with T-cell and dendritic cell responses to viral infection that are significantly higher in mild cases and decreased in expression as severity of COVID-19 increases, were found, suggesting that an immediate and effective activation of T cells is important in modulating disease progression. Together, the findings identify new targets for therapeutic approaches for the treatment of SARS-CoV-2 infection and prevention of complications of severe COVID-19.

In one embodiment, the disclosure relates generally to methods for using biomarkers to determine whether a subject having or is suspected of having SARS-CoV-2 infection is at risk of severe COVID.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

The “amount” or “level” of a biomarker associated with an increased risk of severe COVID, Long COVID or need for admission to the ICU for an individual is a detectable level in a biological or physiological sample. These can be measured by methods known to one skilled in the art and also disclosed herein. The expression level or amount of biomarker assessed can be used to determine the response to the treatment.

The term “biomarker” as used herein refers to an indicator, e.g., predictive, diagnostic, and/or prognostic, which can be detected in a sample. The biomarker may serve as an indicator of a disease or disorder (e.g., severe COVID) characterized by certain, molecular, pathological, histological, and/or clinical features. In some embodiments, a biomarker is a protein or the expression of the gene encoding the protein. Biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers.

The terms “biomarker signature” or “signature” are used interchangeably herein and refer to one or a combination of biomarkers whose expression is an indicator, e.g., predictive, diagnostic, and/or prognostic. The biomarker signature may serve as an indicator of a disease or disorder, or the risk thereof, characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The term “sample” refers to a composition that is obtained or derived from a subject and/or individual of interest that contains a cellular and/or other molecular entity that is to be characterized and/or identified, for example based on physical, biochemical, chemical and/or physiological characteristics. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tissue lysates, and tissue culture medium, tissue extracts such as homogenized tissue, cellular extracts, and combinations thereof.

By “tissue sample” or “cell sample” is meant a collection of similar cells obtained from a tissue of a subject or individual. The source of the tissue or cell sample may be solid tissue as from a fresh ; frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample” or “control sample”, as used herein, refers to a sample, standard, or level that is used for comparison purposes. In one embodiment, a reference sample, reference, or control is obtained from a healthy body of a different subject.

The term “diagnosis” is used herein to refer to the identification or classification of a molecular or pathological state, disease or condition. “Diagnosis” may refer to the classification of a particular disease, e.g., by histopathological criteria, or by molecular features (e.g., characterized by expression of one or a combination of biomarkers).

The term “aiding diagnosis” is used herein to refer to methods that assist in making a clinical determination regarding the presence, or nature, of a particular type of symptom or condition of a disease or disorder. For example, a method of aiding diagnosis of a disease or condition can comprise measuring certain biomarkers in a biological or physiological sample from an individual.

The terms “level of expression” or “expression level” in general are used interchangeably and generally refer to the amount of a biomarker in a biological or physiological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated” expression, “elevated” expression levels or amounts, or “elevated” levels or amounts, or “increased” expression, “increased” expression levels or amounts, or “increased” levels or amounts refer to an increased expression or increased levels of a biomarker in an individual relative to a control, such as an individual or individuals who are not suffering from the disease or disorder, have the disease or disorder but are not in the same category with regard to severity, or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder, have the disease or disorder but are not in the same category with regard to severity, or an internal control (e.g., housekeeping biomarker). In some embodiments, reduced expression is little or no expression.

An “effective amount” of an agent refers to an amount effective, at dosages and for periods of time necessary, to achieve the desired therapeutic or prophylactic result.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects.

The terms “treat,” “treating,” or “treatment” as used herein, include reducing, alleviating, abating, ameliorating, relieving, or lessening the symptoms associated with a disease, disease sate, or indication in either a chronic or acute therapeutic scenario. Also, treatment of a disease or disease state described herein includes the disclosure of use of such compound or composition for the treatment of such disease, disease state, or indication.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “individual” or “subject” is a mammal. Mammals include, but are not limited to, domesticated animals (eg., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater.

By “increase or enhance” is meant the ability to cause an overall increase of 20%, 30%, 40%, 50%, 60%, 70%, 75%, 80%, 85%, 90%, 95%, or greater.

The phrase “based on” when used herein means that the information about one or more biomarkers is used to inform a diagnosis decision, treatment decision, information provided on a package insert, or marketing/promotional guidance, etc.

The term “mesothelin inhibitor” or “MSLN inhibitor” refers to a compound or a molecule that inhibit MSLN synthesis and/or activity. For example, MORAB-009 (Amatuximab) is a chimeric (human-mice) antibody with high affinity to human mesothelin and may be employed as a MSLN inhibitor. Other MSLN inhibitors include, but are not limited to, wheat germ agglutinin, etythroagglutinatin, phytohemagglutinin, or anetumab ravtansine (ARav).

With regard to “antibody, one of ordinary skill in the art will appreciate that an antibody consists of four polypeptides: two identical copies of a heavy (H) chain polypeptide and two copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. The variable regions of each pair of light and heavy chains form the antigen binding site of an antibody. The nucleic acid sequence which encodes an antibody that binds to a ligand on can comprise one or more nucleic acid sequences, each of which encodes one or more of the heavy and/or light chain polypeptides of an antibody. In this respect, the nucleic acid sequence which encodes an antibody that binds to a molecule can comprise a single nucleic acid sequence that encodes the two heavy chain polypeptides and the two light chain polypeptides of an antibody. Alternatively, the nucleic acid sequence which encodes an antibody that binds a molecule can comprise a first nucleic acid sequence that encodes both heavy chain polypeptides of an antibody, and a second nucleic acid sequence that encodes both light chain polypeptides of an antibody. In yet another embodiment, the nucleic acid sequence which encodes an antibody that binds to a molecule, can comprise a first nucleic acid sequence encoding a first heavy chain polypeptide of an antibody, a second nucleic acid sequence encoding a second heavy chain polypeptide of an antibody, a third nucleic acid sequence encoding a first light chain polypeptide of an antibody, and a fourth nucleic acid sequence encoding a second light chain polypeptide of an antibody.

In another embodiment, the nucleic acid sequence encodes an antigen-binding fragment (also referred to as an “antibody fragment”) of an antibody. The term “antigen-binding fragment” refers to one or more fragments of an antibody that retain the ability to specifically bind to an antigen. Examples of antigen-binding fragments include but are not limited to (i) a Fab fragment, which is a monovalent fragment consisting of the V_{L}, V_{H}, C_{L}, and C_{H1} domains; (ii) a F(ab′)2 fragment, which is a bivalent fragment comprising two Fab fragments linked by a disulfide bridge at the hinge region; and (iii) a Fv fragment consisting of the V_{L} and V_{H} domains of a single arm of an antibody. In one embodiment, the nucleic acid sequence which encodes an antibody that binds a molecule, can comprise a nucleic acid sequence encoding a Fab fragment of an antibody.

An antibody, or antigen-binding fragment thereof, can be obtained by any means, including via in vitro sources (e.g., a hybridoma or a cell line producing an antibody recombinantly) and in vivo sources (e.g., rodents). Methods for generating antibodies are known in the art and are described in, for example, Kohler and Milstein, Eur. J. Immunol. 5:511 (1976); Harlow and Lane (eds.), Antibodies: A Laboratory Manual. CSH Press (1988); and C. A. Janeway et al. (eds), Immunobiology, 5th Ed., Garland Publishing, New York, NY (2001)). In certain embodiments, a human antibody or a chimeric antibody can be generated using a. transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9):1117 (2005), and Lonberg, Handb. Exp. Pharmacol., 181:69 (2008)).

The nucleic acid sequence which encodes an antibody that binds to a molecule, or an antigen-binding fragment thereof, can be generated using methods known in the art. For example, nucleic acid sequences, polypeptides, and proteins can be recombinantly produced using standard recombinant DNA methodology (see, e.g., Sambrook et al., Molecular Cloning: A Laboratory Manual, 3rd ed., Cold Spring Harbor Press, Cold Spring Harbor, NY, 2001; and Ausubel et al., Current Protocols in Molecular Biology, Greene Publishing Associates and John Wiley & Sons, NY, 1994). Further, a synthetically produced the nucleic acid sequence which encodes an antibody that binds to a molecule, or an antigen-binding fragment thereof, can be isolated and/or purified from a source, such as a bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified.

The nucleic acid sequence which encodes an antibody that binds to a molecule, may be identified by extracting RNA from the available antibody producing hybridoma cells. cDNA is produced by reverse transcription and PCR amplification of the light and heavy chains and is carried out using a rapid amplification of cDNA ends (RACE) strategy in combination with specific primers for conserved regions in the constant domains.

The nucleic acid sequence which encodes an antibody that binds to a molecule may also be fully or partly humanized by means known in the art. For example, an antibody chimera may be created by substituting DNA encoding the mouse Fc region of the antibody with that of cDNA encoding for human.

The Fab portion of the molecule may also be humanized by selectively altering the DNA of non-CDR portions of the Fab sequence that differ from those in humans by exchanging the sequences for the appropriate individual amino acids.

Alternatively, humanization may be achieved by insertion of the appropriate CDR coding segments into a human antibody “scaffold”.

Resulting antibody DNA sequences may be optimized for high expression levels in mammalian cells through removal of RNA instability elements, a is known in the art.

In an embodiment, a nucleic acid sequence which encodes a polypeptide that forms an antibody, e.g., forms an antibody having two light chains and two heavy chains, a scFV, a chimeric antibody, a single heavy chain and the like, that binds to a molecule, may be expressed under the control of a single promoter, e.g., using a 2A (Chysel) self cleavable sequence between heavy and light chains. The 2A sequence self-cleaves during protein translation and leaves a short tail of amino acids in the C-terminus of the upstream protein. A Furin cleavage recognition site may be added between the 2A sequence and the upstream gene to assure removal of the remaining amino acids. Plasmids expressing the correct inserts may be identified by DNA sequencing and by antibody specific binding using western analysis and ELISA assays.

In an embodiment, a nucleic acid sequence which encodes a polypeptide that forms an antibody that binds to a molecule, may be operably linked to a heterologous signal peptide, e.g., a signal peptide from IL-2, IL-6, CD5, trypsinogen, serum albumin, prolactin, elastin, chymotrypsin, IL-2, trypsinogen-2, or avastin (MKYLLPTAAAGLLLLAAQPAMA (SEQ ID NO:60) or MEFGLSWLFLVAILKGVQC (SEQ ID NO:61)) or a signal peptide from another immunoglobulin (e.g., see Table 1 in Haryadi et al., PloS One. 10:e0116878 (2015), which is incorporated by reference herein) or a homologous signal peptide from an immunoglobulin, expressed under the control of a single promoter, or a light chain and a heavy chain may be expressed from different promoters and may have the same or different signal peptides. Plasmids expressing the correct inserts may be identified by DNA sequencing and by antibody specific binding using western analysis and ELISA assays

### Clinical Status

Patients with SARS-CoV-2 infection can experience a range of clinical manifestations, from no symptoms to critical illness. In general, adults with SARS-CoV-2 infection can be grouped into the following severity of illness categories; however, the criteria for each category may overlap or vary across clinical guidelines and clinical trials, and a patient's clinical status may change over time.

Asymptomatic or presymptomatic infection: Individuals who test positive for SARS-CoV-2 using a virologic test (i.e., a nucleic acid amplification test [NAAT] or an antigen test) but who have no symptoms that are consistent with COVID-19.

Mild illness: Individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.

Moderate illness: Individuals who show evidence of lower respiratory disease during clinical assessment or imaging and who have an oxygen saturation (SpO2)≥94% on room air at sea level.

Severe illness: individuals who have SpO2<94% on room air at sea level, a ratio of arterial partial pressure of oxygen to fraction of inspired oxygen (PaO2/FiO2)<300 mm Hg, a respiratory rate>30 breaths/main, or lung infiltrates>50%.

Critical illness: individuals who have respiratory failure, septic shock, and/or multiple organ dysfunction.

Patients with certain underlying comorbidities are at a higher risk of progressing to severe COVID-19. These comorbidities include being aged ≥65 years; having cancer, cardiovascular disease, chronic kidney disease, chronic liver disease, chronic lung disease, diabetes, advanced or untreated HIV infection, or obesity; being pregnant; being a cigarette smoker; being a transplant recipient; and receiving immunosuppressive therapy.

Initial evaluation for patients may include chest imaging (e.g., X-ray, ultrasound or computed tomography scan) and electrocardiogram. Laboratory testing should include a complete blood count with differential and a metabolic profile, including liver and renal function tests. Although inflammatory markers such as C-reactive protein (CRP), D-dimer, and ferritin are not routinely measured as part of standard care, results from such measurements may have prognostic value.

The definitions for the severity of illness categories listed above also apply to pregnant patients. However, the threshold for certain interventions is different for pregnant patients and nonpregnant patients. For example, oxygen supplementation is recommended for pregnant patients when SpO2 falls below 95% on room air at sea level to accommodate physiologic changes in oxygen demand during pregnancy and to ensure adequate oxygen delivery to the fetus. If laboratory parameters are used for monitoring pregnant patients and making decisions about interventions, clinicians should be aware that normal physiologic changes during pregnancy can alter several laboratory values. In general, leukocyte cell count increases throughout gestation and delivery and peaks during, the immediate postpartum period. This increase is mainly due to neutrophilia. D-dimer and CRP levels also increase during pregnancy and are often higher in pregnant patients than nonpregnant patients.

In pediatric patients, radiographic abnormalities are common and, for the most part, are not the only criteria used to determine the severity of illness. The normal values for respiratory rate also vary with age in children; therefore, hypoxemia should be the primary criterion used to define severe COVID-19, especially in younger children. In a small number of children and in some young adults, SARS-CoV-2 infection may be followed by a severe inflammatory condition called rnultisystem inflammatory syndrome in children (MIS-C).

### Asymptomatic or Presymtpomatic Infection

Asymptomatic SARS-CoV-2 infection can occur, although the percentage of patients who remain truly asymptomatic throughout the course of infection is variable and incompletely defined. It is unclear what percentage of individuals who present with asymptomatic infection progress to clinical disease. Some asymptomatic individuals have been reported to have objective radiographic findings consistent with COVID-19 pneumonia.

### Mild Illness

Patients with mild illness may exhibit a variety of signs and symptoms (e.g., fever, cough, sore throat, malaise, headache, muscle pain, nausea, vomiting, diarrhea, loss of taste and smell). They do not have shortness of breath, dyspnea on exertion, or abnormal imaging. Most patients who are mildly ill can be managed in an ambulatory setting or at home through telemedicine or telephone visits. No imaging or specific laboratory evaluations are routinely indicated in otherwise healthy patients with mild COVID-19. Older patients and those with underlying comorbidities are at higher risk of disease progression; therefore, health care providers should monitor these patients closely until clinical recovery is achieved.

### Moderate Illness

Moderate illness is defined as evidence of lower respiratory disease during clinical assessment or imaging, with SpO2≥94% on room air at sea level. Given that pulmonary disease can progress rapidly in patients with COVID-19, patients with moderate disease should be closely monitored. If bacterial pneumonia is suspected, administer empiric antibiotic treatment, re-evaluate the patient daily, and de-escalate or stop antibiotics if further testing indicates the patient does not have a bacterial infection.

### Severe Illness

Patients with COVID-19 are considered to have severe illness if they have SpO2<94% on room air at sea level, PaO2/FiO2<300 mm Hg, a respiratory rate >30 breaths/min, or lung infiltrates >50%. These patients may experience rapid clinical deterioration. Oxygen therapy should he administered immediately using a nasal cannula or a high-flow oxygen device. If bacterial pneumonia or sepsis is suspected, administer empiric antibiotics, re-evaluate the patient daily, and de-escalate or stop antibiotics if further testing indicates the patient does not have a bacterial infection.

### Critical Illness

SARS-COV-2 infection can cause acute respiratory distress syndrome, virus-induced distributive (septic) shock, cardiac shock, an exaggerated inflammatory response, thrombotic disease, and exacerbation of underlying comorbidities. Successful clinical management of a patient with COVID-19, as with any patient in the intensive care unit (ICU), includes treating both the medical condition that initially resulted in ICU admission as well as other comorbidities and nosocomial complications.

### Persistent Symptoms and Other Conditions After Acute COVID-19

Some patients may experience persistent symptoms or other conditions after acute COVID-19. Adult and pediatric data on the incidence, natural history, and etiology of these symptoms and organ dysfunction are emerging. However, reports on these data have several limitations, including differing case definitions. In addition, many reports only included patients who attended post-COVID-19 clinics, and the studies often lack comparator groups. The nomenclature for this phenomenon is evolving, and no clinical terminology has been established. It has been referred to as post-COVID-19 condition, post-COVID syndrome, post-acute sequelae of SARS-COV-2, or, colloquially, “long COVID,” and affected patients have been referred to as “long haulers.” MIS-C and multisystem inflammatory syndrome in adults (MIS-A) are serious, postinfectious complications of acute COVID-19.

The CDC has defined post-COVID-19 conditions as new, returning, or ongoing symptoms that people experience ≥4 weeks after being infected with SARS-CoV-2. In October 2021, the World Health Organization published a clinical case definition that described the post-COVID-19 clinical condition as usually occurring 3 months after the onset of COVID-19 with symptoms that last for ≥2 months and cannot be explained by an alternative diagnosis.

### Persistent Symptoms

The prevalence of persistent post-COVID-19 clinical signs and symptoms remains unclear. In a systematic review of 25 observational cohort studies, prevalence varied widely (from 5% to 80%) and likely reflected differences in study population, case definition, and data resources. Another large, systematic review found similar prevalence of post-COVID-19 symptoms 6 months after initial infection between studies from high-income or low- and middle-income countries and between studies in which >60% or <60% of the patients were hospitalized. A prospective study conducted at the University of Washington investigated mostly outpatients with laboratory-confirmed SARS-CoV-2 infection (150 participants had mild illness, 11 had no symptoms, and 11 had moderate or severe disease that required hospitalization). 36 Participants completed a follow-up questionnaire 3 months to 9 months after illness onset; 33% of outpatients and 31% of hospitalized patients reported ≥1 persistent symptom. Persistent symptoms were reported by 27% of the patients aged 18 to 39 years, 30% of those aged 40 to 64 years, and 43% of those aged ≥65 years.

In these and other studies, the most commonly reported nonneurologic, persistent symptoms included fatigue or muscle weakness, joint pain, chest pain, palpitations, shortness of breath, and cough. From January 2020 to April 2021, CDC conducted an internes-based survey of 3,135 noninstitutionalized adults who self-reported receiving either a positive or negative SARS-CoV-2 test result. The study found that fatigue, shortness of breath, and cough were commonly reported symptoms lasting >4 weeks after onset. The prevalence of these symptoms among participants with a positive test result versus the prevalence among participants with a negative test result was 22.5% versus 12% for fatigue, 15.5% versus 5.2% for shortness of breath, and 14.5% versus 4.9% for cough.

Some of the reported symptoms overlap with post-intensive care syndrome symptoms that have been described for patients without COVID-19. Prolonged symptoms and disabilities after COVID-19 have also been reported in patients with milder illness, including outpatients.

Patients who had breakthrough infection after COVID-19 vaccination are less likely to report symptoms that persist ≥28 days than patients with SARS-CoV-2 infection who are unvaccinated.46,47 The COVED Symptom Study, conducted from December 2020 to July 2021, included participants who used a mobile application to report symptoms after breakthrough infections or reinfection. The investigators found that the odds of reporting symptoms for ≥28 days was reduced by about half among participants who received 2 vaccine doses, when compared with participants who received 1 or 0 vaccine doses.

A study of electronic health record data from 59 health care organizations, primarily in the United States, compared the records of people who did not receive any vaccine doses with records of people who received 2 vaccine doses.48 In the 6 months after infection, those who received 2 vaccine doses had a lower risk for some, but not all, long-COVID outcomes, such as fatigue, muscle weakness, loss of sense of smell, or hair loss.

### Cardiopulmonary Injury

A U.S. Department of Veterans Affairs (VA) study of a national health care database compared 153,760 veterans who survived the first 30 days of COVID-19 to contemporary and historical control cohorts that had no evidence of SARS-CoV-2 infection. When compared with the control cohorts, patients with a history of COVID-19 had a greater incidence of postacute cardiovascular outcomes (e.g., cerebrovascular disorder, dysrhythmia, inflammatory heart disease, ischemic heart disease, heart failure, thromboembolic disease) at 12 months. A prospective study of pulmonary function examined longitudinal data from the adult Copenhagen General Population Study and found that pulmonary function declined faster (median 5.6 months) for the 107 patients with mostly mild COVID-19 than for a matched sample from the general population.

### Neuropsychiatric Impairment

Neuropsychiatric impairments have been reported among patients who have recovered from acute COVID-19. Reported persistent neurologic symptoms include headaches, vision changes, hearing loss, impaired mobility, numbness in extremities, restless legs syndrome, tremors, memory loss, cognitive impairment, sleep difficulties, concentration problems, mood changes, and loss of sense of smell or taste.

One study in the United Kingdom administered cognitive tests to 84,285 participants who had recovered from suspected or confirmed SARS-CoV-2 infection. These participants had worse performances across multiple domains than would be expected for people with the same ages and demographic profiles; this effect was observed even among those who had not been hospitalized. However, the study authors did not report when the tests were administered in relation to the diagnosis of COVID-19.

A retrospective cohort study examined the electronic health records of 273,618 patients from 59 health care organizations, primarily in the United States. The study reported that cognitive dysfunction (defined using International Classification of Diseases 10th Revision codes) 3 to 6 months after diagnosis was worse for people with COVID-19 than for people with influenza. Other studies have reported high rates of anxiety and depression among patients who evaluated their psychiatric distress using self-report scales. Reports also show that patients aged ≤60 years experienced more psychiatric symptoms than patients aged >60 years.

### Metabolic Perturbations

There have been reports of new-onset diabetes after COVID-19. A study of a VA national health care database analyzed the records of 181,280 people who survived the first 30 days of COVID-19 and compared them to a contemporary control cohort that had no evidence of SARS-CoV-2 infection. People with a history of COVID-19 had a 40% greater risk of diabetes 12 months after infection than people in the control cohort. A CDC study of people aged<18 years reported that those with a history of COVID-19 had an increased risk of diabetes >30 days after SARS-CoV-2 infection when compared with the risk of those with no history of infection.

Research on persistent symptoms and other conditions after COVID-19 is ongoing, including the National Institutes of Health's RECOVER initiative, which aims to better characterize the prevalence, characteristics, and pathophysiology of post-acute sequelae of SARS-CoV-2 and inform potential therapeutic strategies.

### Methods for Diagnosing a Subject with or at Risk of Severe COVID

Aspects disclosed herein provide methods of diagnosing a subject having or at risk of severe COVID comprising: (a) assaying a level of mesothelin (MSLN) in a biological or physiological sample from the subject; and (b) determining whether the subject has or is at risk of severe COD at least based on a result from (a). Further provided herein are methods for determining a subject having severe COVID, comprising (a) assaying a level of MSLN in a biological or physiological sample from the subject; and (b) determining whether the subject has severe COVID at least based on a result from (a). Further provided herein are methods for determining a subject at risk of severe COVID, comprising (a) assaying a level of MSLN; and (b) determining whether the subject is at risk of severe COVID at least based on a result from (a).

The physiological or biological sample may be obtained directly, or indirectly, respectively, from the subject. In some embodiments, the physiological sample comprises a physiological fluid sample. In one embodiment the physiological sample is a tissue sample. In some embodiments, the physiological sample comprises whole blood, sera or plasma. In some instances, the subject is a mammal. In some instances, the subject is a mouse, rat, monkey or other non-human primate, or rabbit, In some instances, the subject is a human. In some embodiments, the assay used in herein may be an assay comprising polymerase chain reaction (PCR), reverse transcription PCR (RT-PCR), deoxyribonucleic acid (DNA) sequencing, ribonucleic acid (RNA) sequencing, genotyping array, immunohistochemistry, enzyme-linked immunosorbent assay (ELISA), single-molecule array (Sigma), or a combination thereof.

In some embodiments, assaying comprises determining the biological or physiological sample has an elevated expression level of MSLN as compared to a reference sample from a corresponding mammal that is not infected with SARS-CoV-2 or does not have severe COVID. In some embodiments, the reference sample is obtained from a corresponding mammal with mild COVID. In some embodiments, the reference sample is obtained from a corresponding mammal with moderate COVID. In some embodiments, the reference sample is obtained from a corresponding mammal with severe COVID. In some embodiments, the expression level of MSLN is determined by an assay comprising immunohistochemistry, enzyme-linked immunosorbent assay (ELISA), western blot, dot blot, high performance liquid chromatography (HPLC), liquid chromatography-mass spectrometry (LC-MS), immunoprecipitation, immunoelectrophoresis, capillary electrophoresis (CE), mass spectrometry, or fluorophore-assisted carbohydrate electrophoresis, or a combination thereof. Those assays include fluorescence, chemiluminescence and colorimetry based assays.

For example, MSLN protein can be detected by antibodies for immunofluorescence, immunohistochemistry, western blot analysis or for quantification through ELISA. Exemplary antibodies include recombinant anti-mesothelin antibody from Abcarn (#ab196235), this is a rabbit monoclonal antibody that reacts to the human protein and mesothelin (EP140) rabbit monoclonal antibody reacting to human protein from Milipore Sigma (#439R).

MSLN protein can be detected by western blot: which allows for the specific identification and characterization of proteins, in this case mesothelin. Proteins are separated by running by sodium dodecyl sulfate-polyacrylamide gel electrophoresis (SDS-PAGE)-and then electophoretically transferred to a polyvinylidene fluoride (PVDF) membrane which is then incubated with a MSLN-specific antibody. Protein can be semi-quantified. through comparison of the band intensity to that on an internal protein control.

MSLN protein can be detected by ELISA, e.g., detected and quantified by solid phase sandwich ELISA using the human mesothelin quantikine ELISA from R&D Systems (#DMSLN0) (Human Mesothelin Quantikine ELISA Kit DMSLN0: R&D Systems (rndsystems.com)). To carry out an ELISA protein 5 μl plasma (1 in 10 diluted in assay diluent) can be incubated in MSLN antibody pre-coated microplates at room temperature for 2 hours. Washing removes unbound elements, then a MSLN-specific enzyme-linked monoclonal antibody is added. After 2 hours of incubation a substrate is added and incubated for a further 30 minutes prior to using a microplate reader to determine optical density after stopping the development reaction.

MSLN protein can be detected by immunofluorescence using standard procedures for preparation of formalin fixed, paraformaldehyde fixed, or methanol fixed human cells or frozen of paraffin embedded formalin fixed tissues.

MSLN protein can be detected by immunohistochemistry in human tissue sections using standard procedures for preparation of paraffin embedded formalin fixed human tissues.

In some embodiments, a subject diagnosed with severe COVID according to the present methods may be administered a MSLN inhibitor. A MSLN inhibitor can be any compound or molecule that can inhibit MSLN synthesis and/or activity.

In some embodiments, the subject diagnosed with severe COVID according to the methods disclosed herein is administered a therapeutically effective amount of an anti-viral compound. In some embodiments, the anti-viral inhibitor inhibits the expression of SARS-CoV-2. In some embodiments, the anti-viral inhibitor comprises an antibody, or antigen-binding fragment. In some embodiments, the anti-viral inhibitor comprises a protease inhibitor. In some embodiments, the anti-viral inhibitor comprises a ribonucleotide or ribonucleoside. In some embodiments, the anti-viral inhibitor is administered before the MSLN inhibitor. In some embodiments, the anti-viral inhibitor is administered after the MSLN inhibitor. In some embodiments, the methods further comprise administering to the subject a therapeutically effective amount of a steroid.

### Methods for Determining Whether a Subject Has or is At Risk of Developing Long-COVID

Aspects disclosed herein provide methods of determining whether a subject has, or is at risk of developing, long COVID, the method comprising (a) assaying a level of MSLN in a biological or physiological sample from the subject; and (b) determining a whether a subject has, or will develop, long COVID at least based on a result from (a). The physiological or biological sample may be obtained directly, or indirectly, respectively, from the subject. In some embodiments, the physiological sample comprises a physiological fluid sample In some embodiments, the physiological sample is a tissue sample. In some embodiments, the physiological sample comprises whole blood, sera or plasma. In some instances, the subject is a mammal. In some instances, the subject is a mouse, rat, monkey, or rabbit. In some instances, the subject is a human. In some embodiments, the assay used in herein may be an assay comprising polymerase chain reaction (PCR), reverse transcription PCR (RT-PCR), deoxyribonucleic acid (DNA) sequencing, ribonucleic acid (RNA) sequencing, genotyping array, immunohistochemistry, enzyme-linked immunosorbent assay (ELISA), single-molecule array (Simoa), or a combination thereof.

In some embodiments, assaying comprises determining the biological. or physiological sample has an elevated expression level of MSLN as compared to a reference sample that does not have COVID or does not have severe COVID. In some embodiments, the reference sample is obtained from a healthy subject or individual. In some embodiments, the expression level of MSLN is determined by an assay comprising immunohistochemistry, enzyme-linked immunosorbent assay (ELBA), western blot, dot blot, high performance liquid chromatography (HPLC), liquid chromatography-mass spectrometry (LC-MS), immunoprecipitation, immunoelectrophoresis, capillary electrophoresis (CE), mass spectrometry, or fluorophore-assisted carbohydrate electrophoresis, or a combination thereof. Those assays include fluorescence, chemiluminescence and colorimetry based assays.

Aspects disclosed herein provide methods of evaluating a biological or physiological sample obtained from the subject for the presence, absence, and/or quantity of a nucleic acid sequence from a gene of the biomarker disclosed herein, or a gene product expressed from the gene of the biomarker disclosed herein. In some cases, the nucleic acid sequence comprises deoxyribonucleic acid (DNA). In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA., a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, are nucleic acid-based detection assays useful for the detection of a presence, absence, and/or quantity of a nucleic acid sequence from a gene of the biotnarker disclosed herein, or gene product expressed from the gene of the biomarker disclosed herein. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytocheinistry, or sequencing. In some embodiments, the sequencing technique comprises next generation sequencing. In some embodiments, the methods involve a hybridization assay such as fluorogenic qPCR (e.g., TaqMan™ or SYBR green), which involves a nucleic acid amplification reaction with a specific primer pair, and hybridization of the amplified nucleic acid probes comprising a detectable moiety or molecule that is specific to a target nucleic acid sequence. An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genes disclosed herein is used. In some instances. the nucleic acid probe is specific to the gene of the biomarker disclosed herein, or gene product expressed from the gene of the biomarker disclosed herein. Disclosed herein, in some embodiments, are methods of detecting a gene of an individual by subject a sample obtained from the individual to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genes, or gene expression products thereof, disclosed herein. In some instances, the amplification assay requires primers. The nucleic acid sequence for the gene, or gene expression products thereof, known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). It will be apparent to one skilled in the art that controlled robotic systems are useful for isolating and amplifying nucleic acids and can be used.

In some embodiments, detecting the presence or absence and/or quantity of a gene of the biomarker disclosed herein, or gene expression produced expressed from the gene of the biomarker disclosed herein, comprises sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Niaxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25. 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides. about 30 to about 100 nucleotides. about 50 to about 100000 nucleotides. about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some embodiments, detecting the presence or absence and/or quantity of a gene of the biomarker disclosed herein, or gene expression produced expressed from the gene of the biomarker disclosed herein, comprises hybridizing a probe or reporting sequence to a target nucleic acid described herein. Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroarotnatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodarnine dyes. Fluorescein and. rhodamine dyes include, but are not limited to 6-carboxyfluorescein (FAM), 2′7′-dimethoxy-4′5′-dichloro-6-carboxyfluorescein (JOE), tetrachlorotluorescein (TET), 6-carboxyrhodamine (R6G), N-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamine compounds include 1-dimethylaminonaphthyl-5-sulfonate, 1-anilino-8-naphthalene sulfonate and 2-p-toluidinyl-6-naphthalene sulfonate, 5-(2′-aminoethypaminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cvanines, such as, e.g., indodicarbocyanine 3 (Cy3), indodicarbocyanine 5 (Cy5), indodicarbocyanine 5.5 (Cy5.5), 34-carboxy-pentyl)-3′-ethyl-5,51-dimethyloxacarbocyanine (CyA); 1H, 5H, 1tH, 15H-Xantheno[2,3,4-ij: 5,6,7-i′j′]diquinolizin-18-ium, 9-[2 (or 4)-[[[642,5-dioxo-1-pyrrolidinyl)oxy]-6- oxohexyl]amino sulfonyl]-4 (or 2)-sulfophenyl]-2,3,6,7,12,13,16,17-octahydro-inner salt (TR or Texas Red); or BODIPYTM dyes. In some cases, the probe comprises FAM as the dye label.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, detecting and quantifying soluble protein levels of a biomarker disclosed herein in a subject by detecting and quantifying the levels from a biological sample obtained from the subject are provided. The biomarker may be detected by use of an antibody-based assay, where an antibody specific to a biomarker disclosed herein (e.g., anti-MSLN antibodies) is utilized. For antibody-based detection methods, the antibody may bind to any region of the biomarker disclosed herein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay.

In some embodiments, MSLN protein may be detected by detecting binding between MSLN and binding partners of MSLN. Methods of analysis of binding between two molecules, as well as with other binding partners comprise performing an assay in vivo or in vitro, or ex vivo. In some instances, the assay may comprise co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

In some embodiments, MSLN can be detected or a level of MSLN can be determined using one of a variety of well known assays based on MSLN-binding proteins or anti-MSLN antibodies, or by quantitation of purified MSLN. MSLN-binding proteins, for example, can be useful in detecting MSLN; a radiometric assay for MSLN based on 125I-labelled MSLN-binding protein. Assays for detecting MSLN or determining a level of MSLN include a variety of competitive and non-competitive binding assays, for example, competitive binding assays using 125I-labeled MSLN binding protein; competitive binding assays based on alkaline phosphatase labeled-MSLN; and non-competitive binding assays based on peroxidase-labeled proteoglycan or peroxidase-labeled MSLN-binding protein, Assays for detecting MSLN or determining a level of MSLN in a sample can be performed using a variety of immunoassay formats, including radioimmunoassays and enzyme-linked immunoassays. Anti-MSLN antiserum useful in immunoassays can be, for example, affinity purified anti-MSLN antiserum

### Exemplary Mesothelin Sequences

Mesothelin is a 40-kDa cell glycosylphosphatidyl inositol (GPI)-linked protein, found in mesothelial cells lining the peritoneum, pleura and pericardium. Mesothelin binds to MUC16/CA125 through a high affinity, N-Glycan dependent interaction. MUC16 is highly expressed on airway epithelial cells which are the primary site of airway infection by SARS-CoV-2.

Mesothelin amounts or levels may be detected by any method. In one embodiment, the mesothelin is human mesothelin. In one embodiment, the mesothelin is soluble mesothelin In one embodiment, the mesothelin to be detected includes but is not limited to the mature protein of

(SEQ ID NO: 1)
malptarpll gscgtpalgs llfllfslgw vqpsrtlage tgqeaapldg vlanppniss
lsprqllgfp caevsglste rvrelavala qknvklsteq lrclahrlse ppedldalpl
dlllflnpda fsgpqactrf fsritkanvd llprgaperq rllpaalacw gvrgsllsea
dvralgglac dlpgrfvaes aevllprlvs cpgpldqdqq eaaraalqgg gppygppstw
svstmdalrg llpvlgqpii rsipqgivaa wrqrssrdps wrqpertilr prfrrevekt
acpsgkkare ideslifykk weleacvdaa llatqmdrvn aipftyeqld vikhkldely
pqgypesviq hlgylflkms pedirkwnvt sletlkalle vnkghemspq vatlidrfvk
grgqldkdtl dtltafypgy lcslspeels svppssiwav rpqdldtcdp rqldvlypka
rlafqnmngs eyfvkiqsfl ggaptedlka lsqqnvsmdl atfmklrtda vlpltvaevq
kllgphvegl kaeerhrpvr dwilrqrqdd ldtlglglqg gipngylvld lsmqealsgt
pcllgpgpvl tvlalllast la;
(SEQ ID NO: 2))
malptarpll gscgtpalgs llfllfslgw vqpsrtlage tgqeaapldg vlanppniss
lsprqllgfp caevsglste rvrelavala qknvklsteq lrclahrlse ppedldalpl
dlllflnpda fsgpqactrf fsritkanvd llprgaperq rllpaalacw gvrgsllsea
dvralgglac dlpgrfvaes aevllprlvs cpgpldqdqq eaaraalggg gppygppstw
svstmdalrg llpvlgqpii rsipqgivaa wrqrssrdps wrqpertilr prfrrevekt
acpsgkkare ideslifykk weleacvdaa llatqmdrvn aipftyeqld vlkhkldely
pqgypesviq hlgylflkms pedirkwnvt sletlkalle vnkghemspq apreplpqva
tlidrfvkgr gqldkdtldt ltafypgylc slspeelssv ppssiwavrp qdldtcdprq
ldvlypkarl afqnmngsey fvkiqsflgg aptedlkals qqnvsmdlat fmklrtdavl
pltvaevqkl lgphveglka eerhrpvrdw ilrgrqddld tlglglqggi pngylvldls
mqealsgtpc llgpgpvltv lalllastla
or
(SEQ ID NO: 3)
malptarpll gscgtpalgs llfllfslgw vqpsrtlage tgqeaapldg vlanppniss
lsprqllgfp caevsglste rvrelavala qknvklsteq lrclahrlse ppedldalpl
dlllflnpda fsgpqactrf fsritkanvd llprgaperq rllpaalacw gvrgsllsea
dvralgglac dlpgrfvaes aevllprlvs cpgpldadqq eaaraalqgg gppygppstw
svstmdalrg llpvlgqpii rsipqgivaa wrqrssrdps wrqpertilr prfrrevekt
acpsgkkare ideslifykk weleacvdaa llatqmdrvn aipftyeqld vlkhkldely
pqgypesviq hlgylflkms pedirkwnvt sletlkalle vnkghemspq vatlidrfvk
grgqldkdtl dtltafypgy lcslspeels svppssiwav rpqdldtcdp rqldvlypka
rlafqnmngs eyfvkiqsfl ggaptedlka lsqqnvsmdl atfmklrtda vlpltvaevq
kllgphvegl kaeerhrpvr dwilrqrqdd ldtlglglqg gipngylvld lsmqealsgt
pcllgpgpvl tvlalllast la,
(mature protein has residues 37 to 606)

The invention will be further described by the following non-limiting examples.

### Example 1

This study was designed to investigate the disparity in clinical severity of COVID-19 and the association with aging and ethnicity through proteomic profiling of patient-derived plasma samples collected in the Keck School of Medicine (KSOM) Biospecimen Repository for COVID-19 at the University of Southern California (USC), representing the spectrum of COVID-19 disease severity. Proteomic profiling of plasma from COVID-19 subjects identifies proteins upregulated in severe COVID-19, and 42 proteins associated with protection from severe COVID-19, were identified.

### Methods

### Patient Recruitment

Patient plasma samples were collected from patients seen at the Keck Hospital, Verdugo Hills, and Los Angeles (LA) County Hospital and stored in the University of Southern California (USC) COVID-19 Biospecimen Repository. None of the subjects were vaccinated. Samples were not analyzed for SARS-CoV-2 variant. For this study, patients were assigned anonymized, coded IDs and were grouped 306 according to the following cohort definitions: severe, indicating subjects who were admitted to the ICU for COVID-19 treatment; moderate, indicating subjects who were hospitalized for COVID-19 treatment but who were not admitted to the ICU; mild, indicating subjects who tested positive for SARS-CoV-2 but did not require hospitalization; and control, indicating subjects who tested negative for SARS-CoV-2 upon admission to the ICU for treatment of other severe diseases. Population demographics for these cohorts are summarized in Table 1. Participants were predominantly Hispanic/Latinx (68%), reflecting the demographics of donors available from the source biorepository (57.4% Hispanic/Latinx, https.//sc-ctsi.org/about/covid-19-biorepository). The mean age of participants in this study was 56.1±1.56 years (Table 2).

### Immunophenotyping

Plasma samples were analyzed for protein expression by Olink proximity extension assays (PEA) for quantification of 184 secreted markers. Olink's Target 96 Inflammation and Target 96 Oncology II panels were chosen for the spread of proteins related to immune response. Each panel consisted of 92 proteins each, of which 6 proteins were included on both panels, resulting in 184 proteins in total and 178 unique proteins. In total, 144 samples were analyzed. Samples were determined to fail quality control if internal incubation and detection controls deviated+/−0.3 Normalized Protein eXpression (NPX) value from the median value across all samples. One sample from the severe cohort (sample 1.1), failed both panels and was excluded. Three samples, one from the control cohort (sample 14) and two from the moderate cohort (samples 28.1 and 66.1), failed the Oncology II panel and were excluded from analysis of that panel but were included in the analysis of the Inflammation panel. Seven proteins had NPX values under the protein-specific limit of detection (LOD) in >50% of samples in all cohorts, and were excluded from statistical analysis, leaving 171 unique proteins. The proteins excluded from analysis were IL-2RB, IL-1 alpha, IL-2, beta-NGF, IL-13, IL-33, and IL-4.

### Statistics

Pairwise comparisons between cohorts were conducted using unpaired Student's t-tests, performed for each individual protein included in the analysis panels. P-values resulting from this analysis were adjusted using the Benjamini-Hochberg method. Network analysis was performed using the STRING database (STRING Consortium, version 11.5). Protein-protein connections were assigned a combined “score” by evaluating probabilities of interaction derived from literature and database mining, then mapped according to these scores; full description of STRING analysis is described in von Mering et al. (2005).

TABLE 1
Summary of study subject demographics. Data represents summarized data from a total of 71 subject plasma
samples used in the current study. All data is expressed as a percentage of the total of subjects.

| | | COVID-19 Positive | | | | | | COVID-19 | | | |
|---|---|---|---|---|---|---|---|---|---|---|---|
| | | Severe | | Moderate | | Mild | | Negative | | All Cohorts | |
| | Demographic | (N = 23) | | (N = 22) | | (N = 10) | | (N = 16) | | (N = 71) | |
| Age group | <18 years | 0 | (0%) | 0 | (0%) | 0 | (0%) | 0 | (0%) | 0 | (0%) |
| | 19-35 years | 0 | (0%) | 2 | (9%) | 2 | (20%) | 0 | (0%) | 4 | (6%) |
| | 36-50 years | 3 | (13%) | 5 | (23%) | 4 | (40%) | 5 | (31%) | 17 | (24%) |
| | 51-65 years | 10 | (43%) | 10 | (45%) | 4 | (40%) | 8 | (50%) | 32 | (45%) |
| | >65 years | 10 | (43%) | 5 | (23%) | 0 | (0%) | 3 | (19%) | 18 | (25%) |
| Sex | Female | 8 | (35%) | 7 | (32%) | 4 | (40%) | 8 | (50%) | 27 | (38%) |
| | Male | 15 | (65%) | 15 | (65%) | 6 | (60%) | 8 | (50%) | 44 | (62%) |
| Race | White or Caucasian | 19 | (83%) | 21 | (95%) | 9 | (90%) | 13 | (81%) | 62 | (87%) |
| | Black or African American | 0 | (0%) | 0 | (0%) | 0 | (0%) | 1 | (6%) | 1 | (1%) |
| | Asian | 2 | (9%) | 0 | (0%) | 1 | (10%) | 1 | (6%) | 4 | (6%) |
| | American Indian or Alaska Native | 0 | (0%) | 1 | (5%) | 0 | (0%) | 0 | (0%) | 1 | (1%) |
| | Native Hawaiian or other Pacific Islander | 0 | (0%) | 0 | (0%) | 0 | (0%) | 0 | (0%) | 0 | (0%) |
| | Other race | 0 | (0%) | 0 | (0%) | 0 | (0%) | 0 | (0%) | 0 | (0%) |
| | Unknown race | 2 | (9%) | 0 | (0%) | 0 | (0%) | 1 | (6%) | 3 | (4%) |
| Ethnicity | Hispanic or Latinx | 18 | (78%) | 17 | (77%) | 5 | (50%) | 8 | (50%) | 48 | (68%) |
| | Not Hispanic of Latinx | 5 | (22%) | 5 | (23%) | 5 | (50%) | 7 | (44%) | 22 | (31%) |
| | Unknown ethnicity | 0 | (0%) | 0 | (0%) | 0 | (0%) | 1 | (6%) | 1 | (1%) |

TABLE 2
Summary of top DEPs comparing all subject cohorts. For each pairwise comparison,
total DE indicates the total number of differentially expressed proteins
(DEPs); up indicates the number of DEPs in that comparison that were increased,
down indicates the number of DEPs in that comparison that were decreased.
“Most sig” indicates the most significant DEP in each comparison;
% change and adjusted (adj.) p-value are listed for that DEP.

| Analysis | Total DE | Up | Down | Most sig | % change | Adj. p-value |
|---|---|---|---|---|---|---|
| Severe vs moderate | 15 | 14 | 1 | TNFB | −40.4 | 1.24E−02 |
| Severe vs mild | 101 | 80 | 21 | EN-RAGE | 499.6 | 1.49E−09 |
| Severe vs control | 62 | 62 | 0 | MSLN | 274.0 | 3.41E−06 |
| Mild vs control | 70 | 40 | 30 | TRANCE | 155.7 | 3.63E−05 |
| Mild vs moderate | 47 | 9 | 38 | IL6 | −85.9 | 6.82E−05 |
| Moderate vs control | 28 | 28 | 0 | CXCL1 | 136.8 | 7.34E−03 |

### Results

Blood plasma samples were obtained from the USC COVID-19 Biorepository and were collected from subjects seen at the Keck Hospital (53.5%), Verdugo Hills (12.7%) and Los Angeles County Hospital (33.8%). Table 1 provides the core demographics of the subject population that provided samples for this project. The population of biobank donors was predominantly Hispanic/Latinx (57.4%, https://sc-ctsi.org/about/covid-19-biorepository), with samples unevenly distributed across all categories of COVID-19 severity. In cases requiring hospitalization, >75% of samples were from Hispanic/Latinx subjects. Subjects were segregated into four independent cohorts based on the hospitalization status of the patient. Categories of severe, moderate, mild and control were based on the following cohorts: 1) severe were COVID-19 positive subjects in the intensive care unit (ECU) being treated for COVID-related illness, 2) moderate were COVID-19 positive subjects that were hospitalized but not requiring ICU treatment, 3) mild were COVID-19 positive subjects that did not require hospitalization and 4) control were COVID-19 negative subjects that were treated in the ICU for other severe illness. The mean age of participants in the study across all categories was 56.1±1.56 years. 25% of the subjects were over 65 years and 6% were under 35 years. The mean age within each subject cohort is included in Table 5. Overall, 62% of the subjects were male, 87% were White/Caucasian, and 68% were Hispanic/Latina. For hospitalized COVID-19 patients in the severe and moderate groups, samples were obtained at day of admission (Day 1), Day 3, Day 5, and Day 7, where available. For the COVID-19 negative ICU subjects and mild COVID-19 cohort the only sample evaluated was day of test/admission (Day 1).

TABLE 5
Age distribution of subjects providing plasma samples. Mean
ages were calculated for <18 years (N = 0), 19-35
years (N = 4), 36-50 years (N = 17), 51-65 years
(N = 31), and >65 years (N = 18). Data is expressed as mean ± S.E.M.

| Age Group | Mean Age | SEM |
|---|---|---|
| <18 years | N/A | N/A |
| 19-35 years | 28.50 | 2.33 |
| 36-50 years | 42.82 | 1.31 |
| 51-65 years | 57.84 | 0.74 |
| >65 years | 71.83 | 1.13 |

TABLE 6
Significant differentially expressed proteins (DEPs) between Hispanic
and non- Hispanic subjects providing plasma samples. Data is
collated from 48 Hispanic and 21 non-Hispanic subjects. One subject
with unknown ethnicity is excluded from this analysis.

| | | | % | | adj. |
|---|---|---|---|---|---|
| Assay | OlinkID | UniProt | change | p-value | p-value |
| ADA | OID00560 | P00813 | 64.25 | 6.49E−04 | 0.0365 |
| TSLP | OID00497 | Q969D9 | 43.16 | 7.85E−04 | 0.0365 |
| FASLG | OID00694 | P48023 | −34.78 | 8.34E−04 | 0.0365 |
| CEACAM5 | OID00739 | P06731 | 126.87 | 9.67E−04 | 0.0365 |
| AREG | OID00728 | P15514 | 108.30 | 0.0010 | 0.0365 |
| S100A11 | OID00727 | P31949 | 51.70 | 0.0012 | 0.0365 |
| TRANCE | OID00521 | 014788 | −38.95 | 0.0018 | 0.0467 |
| EN-RAGE | OID00541 | P80511 | 95.18 | 0.0020 | 0.0467 |
| MCP-3 | OID00474 | P80098 | 145.71 | 0.0035 | 0.0637 |
| MIA | OID00701 | Q16674 | −19.46 | 0.0037 | 0.0637 |
| ANXA1 | OID00745 | P04083 | 64.72 | 0.0039 | 0.0637 |
| SYND1 | OID00664 | P18827 | 85.11 | 0.0042 | 0.0637 |
| MCP-1 | OID00484 | P13500 | 40.63 | 0.0094 | 0.1335 |
| PD-LI | OID00518 | Q9NZQ7 | 54.68 | 0.0169 | 0.2037 |
| IL8 | OID00471 | P10145 | 57.62 | 0.0177 | 0.2037 |
| IL-18R1 | OID00517 | Q13478 | 49.00 | 0.0177 | 0.2037 |
| MSLN | OID00660 | Q13421 | 66.60 | 0.0211 | 0.2185 |
| CXCL1 | OID00496 | P09341 | 42.55 | 0.0214 | 0.2185 |
| TNFB | OID00561 | P01374 | −24.48 | 0.0282 | 0.2733 |
| CD8A | OID05124 | P01732 | −27.28 | 0.0323 | 0.2969 |
| FURIN | OID00688 | P09958 | 19.98 | 0.0353 | 0.3092 |
| IL6 | OID00666 | P05231 | 115.36 | 0.0480 | 0.3749 |

Proteomic analysis of the plasma samples was completed using Olink® proximity extension assays (PEA) for quantification of 184 secreted immunoregulatory biomarkers. The Olink Target 96 Inflammation and Target 96 Oncology II panels were chosen for the spread of proteins related to immune response. Each panel consisted of 92 proteins each, of which 6 proteins were analyzed on both panels, resulting in 184 proteins in total and 178 unique proteins. Samples were determined to fail quality control if internal incubation and detection controls deviated +/−0.3 Normalized Protein eXpression (NPX) value from the median value across all samples. One sample, from the severe cohort, failed both panels and was excluded. Three samples, one from the control cohort and two from the moderate cohort, failed only the Oncology II panel and was excluded from analysis of that panel but was included in the analysis of the Inflammation panel. Seven proteins had NPX values under the protein-specific limit of detection (LOD) in >50% of samples in all cohorts, and were excluded from downstream analysis, 107 leaving 171 unique proteins.

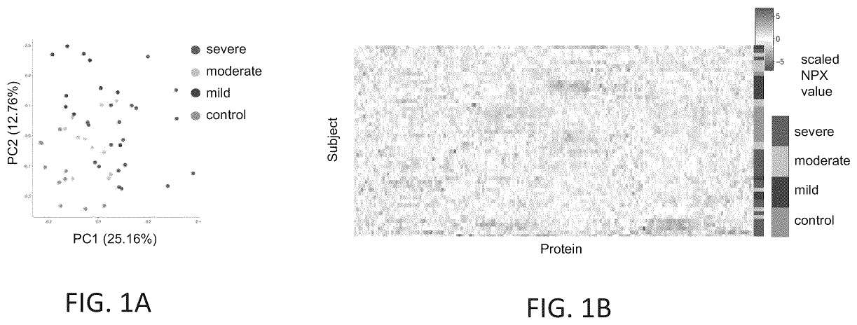

A principal component analysis (PCA) was performed to highlight variation between proteomic signatures of plasma samples collected at Day 1 (FIG. 1A). Samples in the severe COVID-19 cohort clustered together (FIG. 1A, red) and separated from the mild cohort (FIG. 1A, cyan), with samples from patients classified as moderate COVID-19 (FIG. 1A, yellow) straddling these two groups. The control cohort was largely separate from all three COVID-19 cohorts (FIG. 1A, blue). Unbiased clustering by protein NPX values is visualized in the heatmap featured in FIG. 1B. Again, the samples clustered by severity, indicating that protein expression signatures exist representing COVID-19 severity in our patient cohorts. To specifically evaluate changes in protein expression and their association with COVID-19 disease severity, pairwise comparisons of the mean NPX values for each protein were used to determine differentially expressed proteins (DEPs) between subject cohorts. Of the 171 unique proteins analyzed, 101 DEPs were observed between our study cohorts. 118 DEPs between each study cohort are summarized in Table 2, and a comprehensive list of the significant DEPs across all study cohorts, as determined by unpaired Student's t-tests of NPX value by cohort, is available in the full data set.

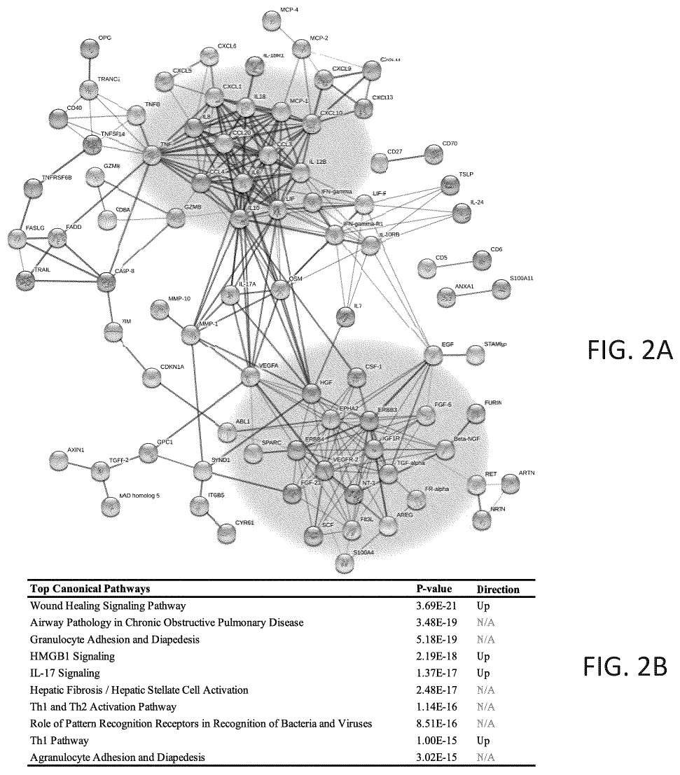

To establish the identity of the proteomic signatures associated with the severity of a subject's response to SARS-CoV-2 infection, DEPs between subject cohorts were compared. To evaluate the biological processes associated with the DEPs, STRING network analysis and Ingenuity Pathway Analysis (IPA) focusing on DEPs significantly different between severe COVID-19 compared to all other cohorts were performed. Two major “hubs” of protein interactions were observed. The first centered around proteins associated with activation of both Th1 and pro-inflammatory cytokines and chemokines including tumor necrosis factor beta (TNFβ), interleukin-6 (IL-6), interleukin-8 (IL-8), interleukin-12 (IL-12), CXC motif chemokine ligand 9 (CXCL9), and CC motif chemokine ligand 3 (CCL3), as well as Th2 and anti-inflammatory cytokines and chemokines including interleukin-10 (IL -10), thymic stromal lymphopoietin (TSLP), and CC motif chemokine ligand 20 (CCL20) (FIG. 2A). Interleukin-17 (IL-17) signaling and Th1 and Th2 activation pathways were among the most significantly changed pathways specific to severe COVID-19 (FIG. 213). The second clustered around growth factors and growth factor receptors including fibroblast growth factor 5 (FGF-5), colony stimulating factor (CSF), ephrin typa-A receptor 2 (EPHA2), protransforming growth factor alpha (TGF-α), and beta-nerve growth factor (β-NGF), suggesting significant activation of tissue regeneration, differentiation and survival signaling pathways (FIG. 2A). IPA also identified wound healing and airway pathologies associated with chronic lung disease as some of the most significantly changed pathways specific to severe COVID-19 (FIG. 2B). In conjunction with tissue remodeling, we also identified connections between Fas-associated death domain protein (FADD), Fas ligand (FASLG), TNF-related apoptosis-inducing ligand (TRAIL), and caspase 8 (CASP-8), markers associated with apoptosis. IPA also validated the upregulation of a pathogenic response including granulocyte and agranulocyte adhesion, diapedesis, and pattern recognition receptor pathways (FIG. 2B).

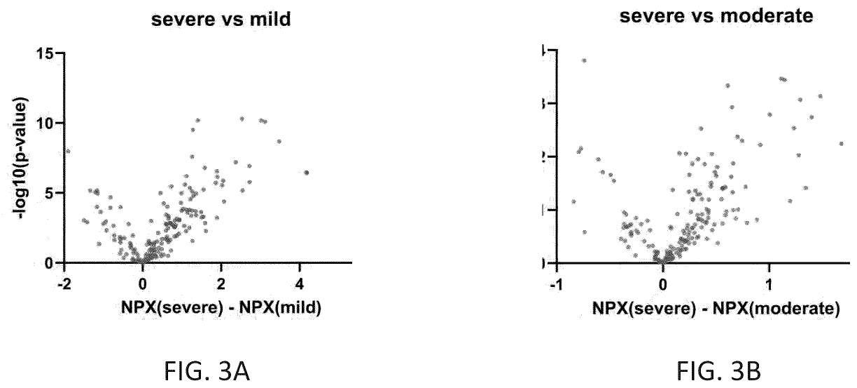

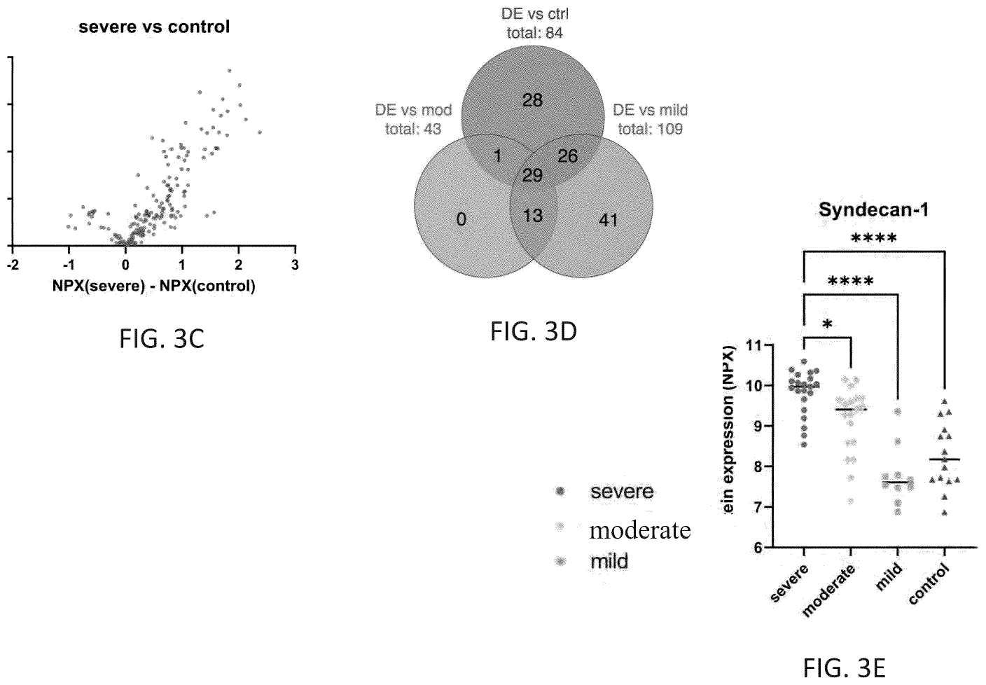

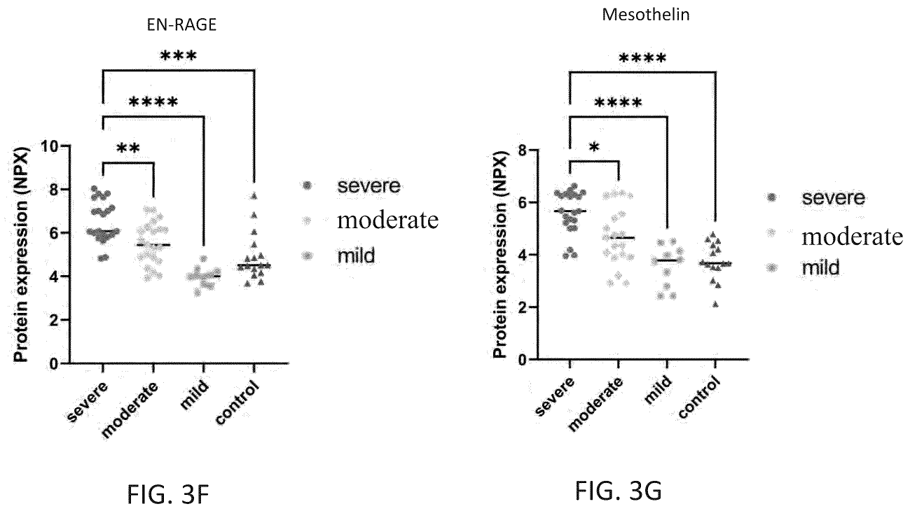

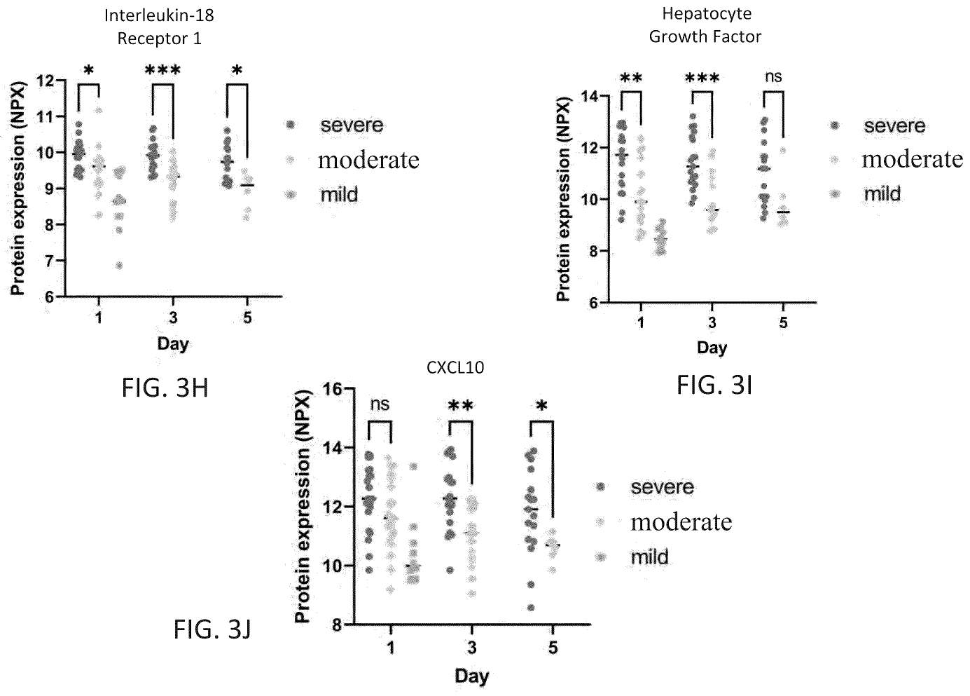

Volcano plots highlighting significant DEPs between the severe COVID-19 cohort and each of the other subject cohorts show 101 DEPs comparing the severe and mild COVID-19 cohorts (FIG. 3A), 15 DEPs comparing the severe and moderate COVID-19 cohorts (FIG. 3B), and 62 DEPs comparing the severe and control 144 cohorts (FIG. 3C). To determine a protein signature specific to severe COVID-19, DEPs between all paired analyses represented in the volcano plots were overlaid in a Venn diagram (FIG. 3D). In total, 10 DEPs were significant in all comparisons: EN-RAGE, WFDC2, AREG, WISP-1, CEACAM5, TNFRSF6B, SYND1, MSLN, PD-L1, and S100A4 (Table 3). Plots representing the samples included in each cohort highlight a severity-associated decline in the amount of plasma proteins detected, as shown for syndecan-1 (SYND1, FIG. 3E), EN-RAGE (S100A12, FIG. 3F), and mesothelin (MSLN, FIG. 3G).

TABLE 3
DEPs significant to severe COVID-19 versus all other cohorts. List of DEPs that were significant
when comparing the severe cohort (N = 23) to mild (N = 10), moderate (N = 22),
and control (N = 16) cohorts. % change and adjusted (adj.) p-value are listed for each pairwise comparison.

| | | | severe vs mild | | severe vs moderate | | severe vs control | |
|---|---|---|---|---|---|---|---|---|
| Assay | OlinkID | UniProt ID | % change | Adj. p-value | % change | Adj. p-value | % change | Adj. p-value |
| EN-RAGE | [symbol] | [symbol] | 499.6 | 1.49E−09 | 108.1 | 2.08E−02 | 213.4 | 3.88E−04 |
| [symbol] | [symbol] | [symbol] | [symbol] | 2.14E−09 | 55.2 | 1.39E−02 | [symbol] | 4.38E−04 |
| AREG | [symbol] | P15514 | 444.9 | 5.72E−07 | 156.8 | 1.39E−02 | 325.2 | 5.08E−06 |
| CEACAM5 | [symbol] | P06731 | 583.4 | 7.92E−07 | 189.1 | 1.39E−02 | 352.8 | 3.98E−05 |
| WISP-1 | [symbol] | O95388 | 205.0 | 7.92E−07 | 119.9 | 1.24E−02 | 115.3 | 4.70E−04 |
| [symbol] | [symbol] | O95407 | 287.2 | 1.91E−06 | 130.1 | 1.24E−02 | 79.2 | 0.0338 |
| SYND1 | [symbol] | P18827 | 322.0 | 1.25E−05 | 65.0 | 0.4471 | 197.6 | 3.58E−05 |
| MSLN | [symbol] | Q13421 | 325.1 | 1.57E−05 | 97.0 | 0.04913 | 274.0 | 3.41E−06 |
| PD-L1 | [symbol] | Q9NZQ7 | 130.8 | 5.20E−04 | 59.2 | 2.08E−02 | 235.3 | 2.34E−05 |
| S100A4 | [symbol] | P26447 | 14.8 | 0.0414 | 30.3 | [symbol] | 60.7 | 2.39E−04 |

[symbol]

indicates data missing or illegible when filed

Interestingly, several proteins that were elevated in both severe and moderate COVID-19 on Day 1 differentially resolved over time in the moderate COVID-19 cohort, lowering to levels comparable to the mild cohort by Day 5 while remaining elevated in the severe COVID-19 cohort. These proteins included interleukin-18 receptor 1 (IL18-R1, FIG. 3H), hepatocyte growth factor (HGF, FIG. 3I), and CXC motif chemokine ligand 10 (CXCL10, FIG. 3J). Such changes in the signaling pathways associated with these proteins likely underly e timely resolution of COVID-19 disease.

Ethnicity was established as a risk factor for severe COVID-19 early during the pandemic, following the observation of higher levels of hospitalization and more severe disease outcomes for Hispanic compared to non-Hispanic subjects (Renelus et al., 2021; Chiumento et al., 2022). Given the ethnic disparity in the Los Angeles community, the samples in our cohort were majority Hispanic/LatinX (68%, Table 1), providing an opportunity to evaluate the intersection of ethnicity and severity as characterized by proteomic profiling. 13 significant DEPs were identified between Hispanic and non-Hispanic subjects (Table 6). All 13 of these DEPs were also significantly differentially expressed between severe cohort and at least one other cohort, 12 were significant between severe and mild cohorts, 9 between severe and control cohorts and 5 between severe and moderate cohorts. Amphiregulin (AREG), CEA cell adhesion molecule 5 (GEACAM5), SYND1, EN-RAGE, and MSLN are all significant DEPs segregating Hispanic from non-Hispanic and severe COVID-19 from all other cohorts (Table 6). Disparity in disease outcome has also been associated with sex as a biological variable with males overrepresented in cases having severe complications from COVID-19 (Mikami et al., 2021). The subject population comprised of 62% male and 38% female subjects. While the control cohort was evenly split, with 50% male and 50% female subjects, all COVID-19 cohorts were predominantly male (severe 65%, moderate 65% and mild 60%). Comparing NPX values between male and female subjects we observed no significant DEPs.

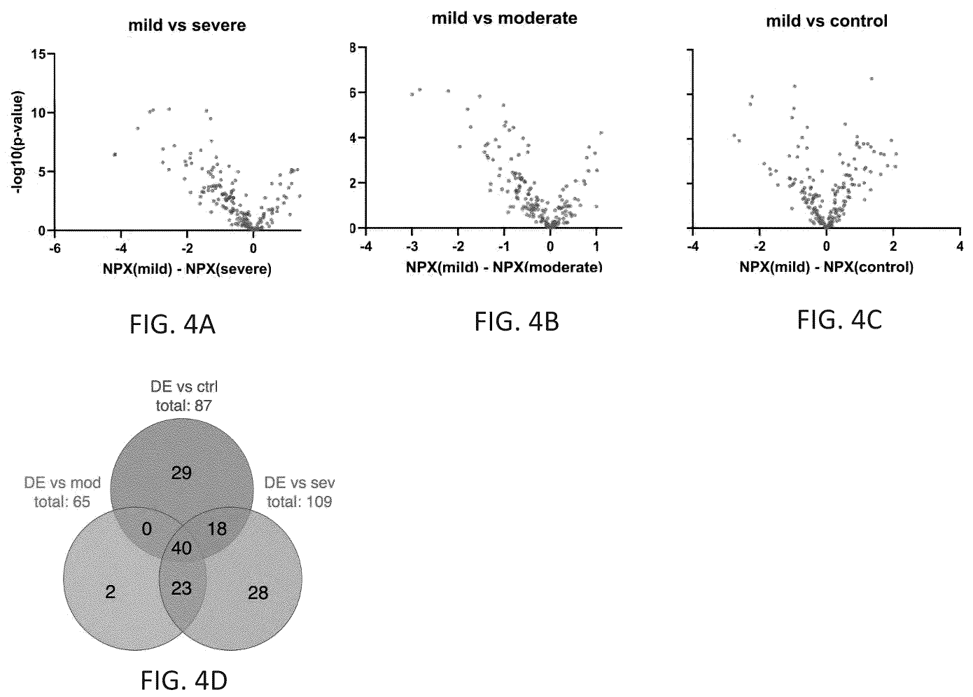

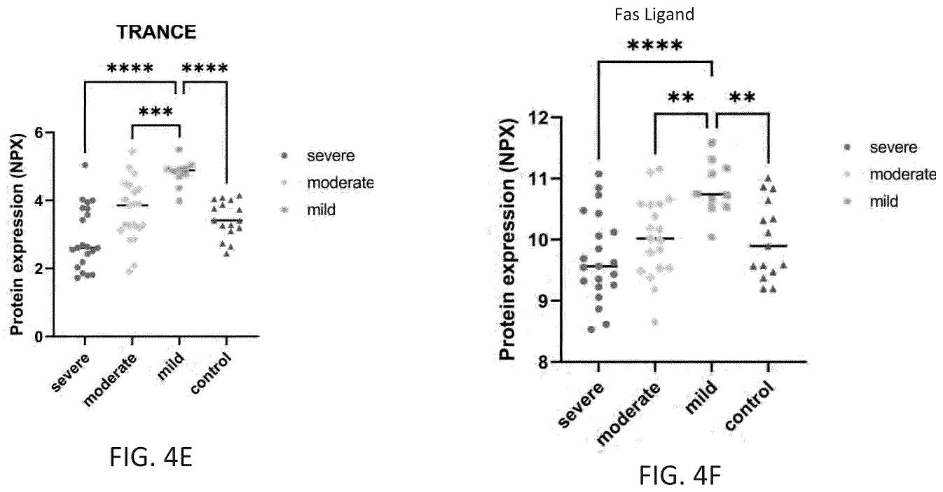

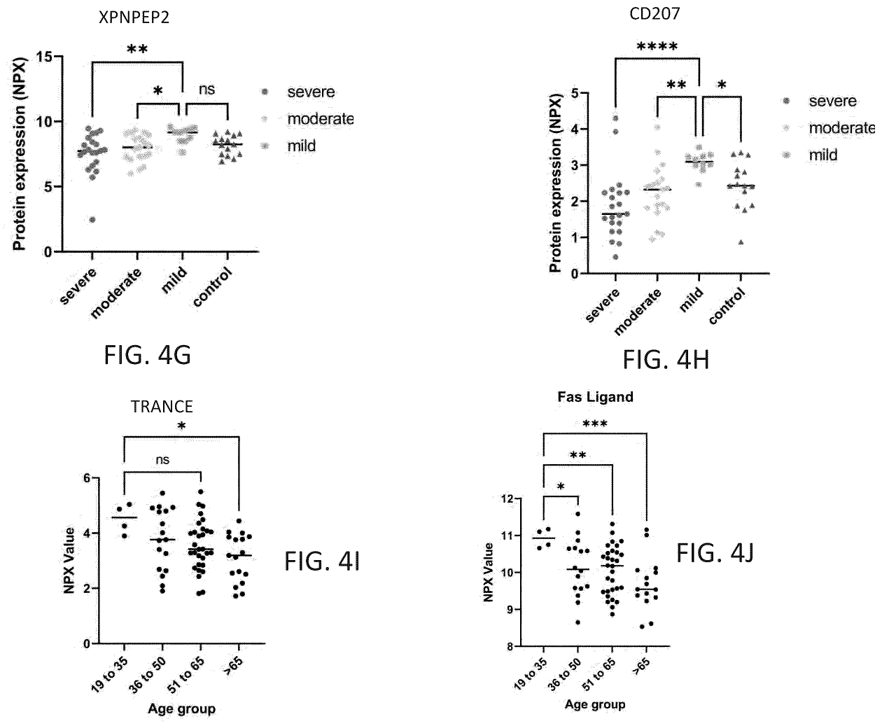

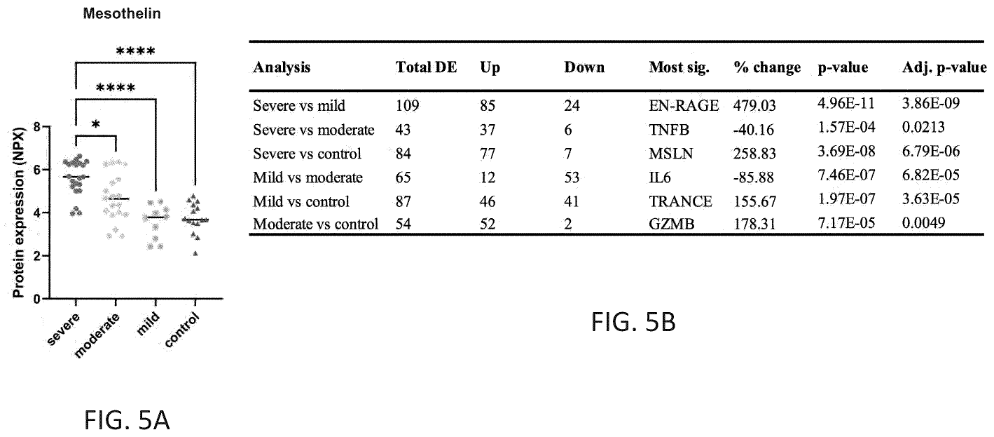

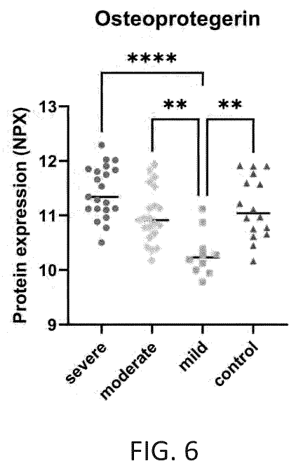

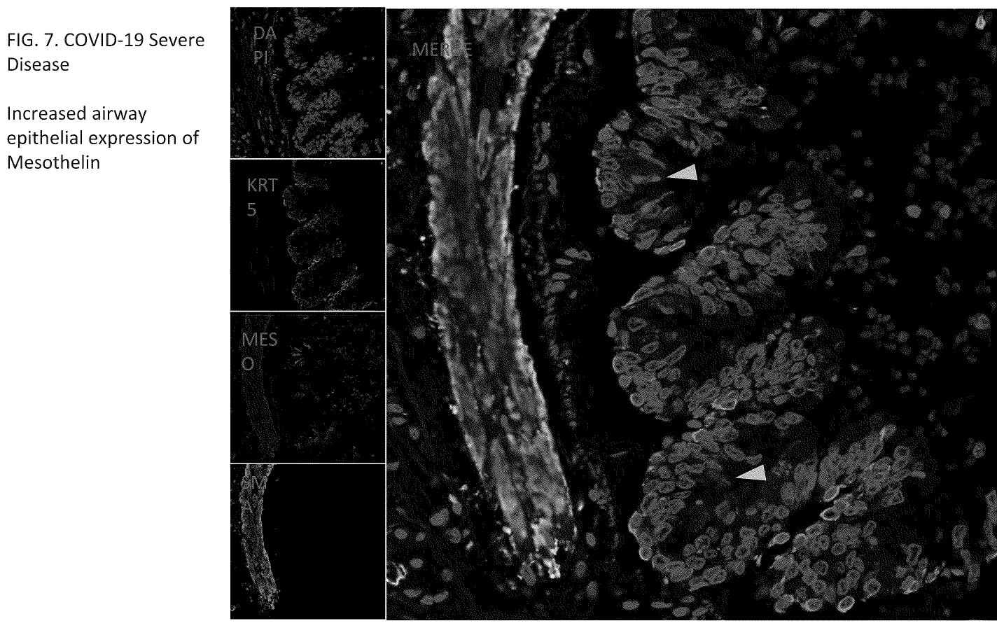

In search of biomarkers potentially associated with an efficient and “protected” response to SARS-CoV-2 infection DEPs unique to the mild COVID-19 cohort were identified. In total, 101 significant DEPs were identified comparing the mild to severe COVID-19 cohorts (FIG. 4A), 47 comparing the mild to moderate COVID-19 cohorts (FIG. 4B), and 70 comparing the mild to control cohorts (FIG. 4C). Overlaying these DEPs in the Venn diagram in FIG. 4D highlights 27 proteins specific to mild COVID-19 (Table 4). Most of these proteins were detected at significantly lower abundance in the mild cohort (78%, Table 4), likely representative of a tempered immune response and decreased persistence of pro-inflammatory cytokines and chemokines. 5 DEPs were significantly augmented in the mild subject cohort and downregulated with severity of COVID-19. These proteins included TNF superfamily member 11 (TRANCE aka RANK-L, FIG. 4E), FASLG (FIG. 4F), XPNPEP2 (FIG. 4G), and CD207 (FIG. 4H). Expression of these proteins may be associated with a stronger response to fighting infection with SARS-CoV-2. In addition, osteoprotegerin (OPG), a decoy receptor for TRANCE, was more highly expressed in the severe cohort (FIG. 7). Interestingly, two of these proteins (TRANCE and FASLG) were also expressed at higher levels in the youngest patient cohort (19 to 35 years) decreasing in expression with age (FIGS. 4I and 4J). This finding is consistent with age being a predominant co-morbidity associated with COVID-19 (Mikami et al., 2021; Suleyman et al., 2022) and suggests that circulating levels of TRANCE and FASLG may be associated with a more effective response to SARS-CoV-2 infection.

TABLE 4
DEPs significant to mild COVID-19 versus every other cohort. List of DEPs that were significant
when comparing the mild cohort (N = 10) to severe (N = 23), moderate (N = 22),
and control (N = 16) cohorts. % change and adjusted (adj.) p-value are listed for each pairwise comparison.

| | | | mild vs severe | | mild vs moderate | | mild vs control | |
|---|---|---|---|---|---|---|---|---|
| Assay | OlinkID | UniProt ID | % change | Adj. p-value | % change | Adj. p-value | % change | Adj. p-value |
| HGF | [symbol] | P14210 | −88.3 | 1.49E−09 | −71.1 | 1.67E−04 | −78.5 | [symbol] |
| [symbol] | [symbol] | P80511 | −83.3 | 1.40E−09 | −65.3 | 6.82E−05 | −47.7 | 2.27E−02 |
| WFDC2 | [symbol] | Q14508 | −62.9 | 2.14E−09 | −42.4 | 6.48E−04 | −39.7 | 1.41E−03 |
| TGF-alpha | [symbol] | [symbol] | −59.4 | 2.30E−09 | −48.6 | 5.26E−04 | −48.1 | 3.90E−05 |
| MCP-3 | [symbol] | P80098 | −91.4 | 1.61E−08 | −78.4 | 6.82E−05 | −53.7 | 2.09E−02 |
| TRANCE | [symbol] | O14788 | 274.4 | 9.62E−08 | 115.2 | 9.33E−04 | 155.7 | 3.63E−05 |
| TNFRSF6B | [symbol] | O95407 | −74.2 | 1.91E−06 | −40.6 | 2.85E−02 | −53.7 | 3.87E−03 |
| IL6 | [symbol] | P05231 | −94.3 | 2.49E−06 | −85.9 | 6.82E−05 | −85.2 | 1.38E−03 |
| IL8 | [symbol] | P10145 | −74.3 | 4.47E−06 | −62.7 | 3.63E−03 | −52.8 | 2.24E−02 |
| MUC-16 | [symbol] | [symbol] | 74.4 | 9.67E−06 | −62.1 | 2.36E−03 | −69.0 | 1.07E−02 |
| [symbol] | [symbol] | O00300 | −53.49 | 1.57E−05 | −37.99 | 4.35E−03 | −44.38 | 2.15E−03 |
| TNFSF13 | [symbol] | O75888 | −40.38 | 1.59E−05 | −28.5 | 3.63E−03 | −33.1 | 2.17E−03 |
| EPHA2 | [symbol] | P29317 | −58.90 | 2.23E−05 | −34.7 | 2.83E−02 | −27.9 | 4.42E−02 |
| CD207 | [symbol] | Q9UJ71 | 129.68 | 3.05E−05 | 79.7 | 2.36E−03 | 54.4 | 1.65E−02 |
| ESM-1 | [symbol] | Q9NQ30 | −50.3 | 3.07E−05 | −32.0 | 3.33E−02 | −49.0 | 1.55E−04 |
| CTSV | [symbol] | O60911 | 153.2 | 3.58E−05 | 95.9 | 3.85E−03 | 136.2 | 1.90E−03 |
| FASLG | [symbol] | P48023 | 124.2 | 3.73E−05 | 73.6 | 5.28E−03 | 78.9 | 4.96E−03 |
| OSM | [symbol] | P13725 | −82.4 | 4.74E−05 | −74.3 | 2.36E−03 | −64.9 | 1.24E−02 |
| CXCL9 | [symbol] | Q07325 | −76.8 | 1.08E−04 | −61.3 | 4.55E−03 | −50.7 | 3.47E−02 |
| CSF-1 | [symbol] | [symbol] | −31.8 | 2.17E−04 | −27.9 | 2.36E−03 | −32.8 | 0.0007 |
| LIF-R | [symbol] | P42702 | −35.7 | 3.14E−04 | −33.5 | 1.50E−03 | −32.5 | 1.07E−02 |
| IL-17C | [symbol] | Q9P0M4 | −62.5 | 4.14E−04 | −53.5 | 2.36E−03 | −38.7 | 0.0491 |
| IL10 | [symbol] | P22301 | −66.8 | 8.20E−04 | [symbol] | 7.75E−04 | −45.2 | 4.20E−03 |
| CCL20 | [symbol] | P78556 | −64.3 | 1.44E−03 | [symbol] | 5.28E−03 | −68.5 | 1.64E−02 |
| XPNPEP2 | [symbol] | O43895 | 161.28 | 2.68E−03 | 102.11 | 1.63E−02 | 70.2 | 3.43E−02 |
| IL-17A | [symbol] | Q16552 | −36.40 | 7.03E−03 | −50.45 | 0.0001 | −36.7 | 0.041 |
| EGF | [symbol] | P01133 | 61.40 | 0.025 | 68.58 | 4.13E−02 | 120.0 | 1.07E−02 |

[symbol]

indicates data missing or illegible when filed

### Discussion

One of the major challenges in managing the COVID-19 pandemic is the wide variation in disease severity, ranging from mild respiratory symptoms that resolve with minimal outpatient treatment to acute respiratory distress syndrome (ARDS) requiring ICU admission and mechanical ventilation. The phenomena, underlying this disparity in disease progression are still poorly understood. In this study, we present data evaluating plasma protein expression that characterizes COVID-19 disease response by severity, evaluating proteins beyond the typical analysis of inflammation associated proteins. The signatures identified in our study , include several proteins that have been previously associated with severe COVID-19 in both clinical datasets and in vitro models, as well as several new markers that have previously not been associated with the severity of response to SARS-CoV-2 infection. In severe COVID-19, the most significant and highly enriched proteins were associated with an inflammatory response to viral infection, including IL-6, IL-8, CCL3, CXCL9, and CXCL10. Several of these have ongoing clinical trials evaluating the effectiveness of 198 regulating these proteins in preventing progression to severe COVID-19 and associated ARDS. Furthermore, our data correlates with previous findings showing a COVID-19-specific elevation of inflammatory signaling pathways (Song et al., 2020a). SYND1 and EN-RAGE, markers associated with COVID-19 disease severity and mortality (Maldonado et al., 2022; Zhang et al., 2021; Russell et al., 2022; Zeng et al., 2021) were among the most significantly elevated markers in our severe cohort.

SYND1 is a heparan sulfate proteoglycan, involved in leukocyte recruitment, vascular repair, and tumor angiogenesis (Tong et at, 2012). Increased expression of this marker is associated with acute endothelial glycocalyx degradation, as well as higher oxygen and mechanical life support requirements and mortality from COVID-19 (Ogawa et al., 2021; Dupont et al., 2021; Karampoor et al., 2021; Johansson et al., 2011). In addition to its significance as a potent marker of endothelial damage during the pathogenesis of COVID-19 (Stahl et al., 2020), both facilitation of viral entry via ACE2 co-localization (Hudak et al., 2021) and transmission of virus to epithelial cells via binding with dendritic cells (Bermejo-Jambrina et al., 2021) have been proposed as mechanisms by which SYND1 actively modulates SARS-CoV-2 infection, making it an important candidate as both a biomarker and a potential target for therapeutic intervention.

EN-RAGE is a pro-inflammatory calcium-binding protein associated with a variety of inflammatory conditions across organ systems, including rheumatoid and psoriatic arthritis, coronary heart disease, cystic fibrosis, autoimmune hepatitis, and Kawasaki disease (Foell et al., 2003a; Ligthart et al., 2004; Foell et al., 2003b; Wu et al., 2020; Foell et al., 2003c). Elevated levels of EN-RAGE have been shown to correlate to increased inflammation in response to COVID-19 (Thwaites et al., 2021), including in asymptomatic cases up to 8 months after infection (Tserel et al., 2021). It has also been found elevated in the blood plasma of subjects with chronic obstructive pulmonary disease (COPD) versus healthy controls (Acevedo et al., 2021), a disease which is associated with higher risk of hospitalization, ICU admission, and mortality from COVID-19 (Gerayeli et al., 2021). EN-RAGE has been explored in the context of COVID-19 in connection with other major inflammatory mechanisms that have been implicated in COVID-19 disease severity, including the renin-angiotensin system (Chiappalupi et al., 2021a; Chiappalupi et al., 2021b), T-cell associated cytokines (Luo et al., 2021), and a dysregulated macrophage population (Chen et al., 2022). The variety of mechanisms that EN-RAGE intersects with suggests that further experiments are needed to define its precise role in increasing COVID-19 severity.

In addition to inflammatory pathways which have been previously reported and an ongoing focus of research to target the “cytokine storm” associated with progression to ARDS (Hu et al., 2020; Song et al., 2020b; Wang et al., 2020))), our analysis also identified a cluster of growth factors and associated proteins that are significantly and specifically elevated in severe COVID-19, including FGF-5, CSF, EPHA2, TGF-α, and β-NGF. These proteins have known roles in cell proliferation of multiple tissue types, including hair growth (Higgins et al., 2014), macrophage and monocyte cell populations (Ushach & Zlotnik, 2016), lymphatic endothelial cells (Yoshimatsu et al., 2020), and the central nervous system (Iulita & Cuello, 2016), as well as various cancers (Bragado et al., 2020; Cannarile et al., 2017; Feng et al., 2020; Li et al., 2018; Li et al., 2016), suggesting a dysregulated attempt to repair tissues in response to damage caused by viral infection. Fatal COVID-19 is marked by severe pulmonary damage, diffuse alveolar damage, in conjunction with endotheliitis and microthrombosis (Bosmuller et al., 2021; Ostergaard, 2021). This damage is compounded by a dysregulated and ineffective attempt at tissue repair, with loss of basal cell populations, squamous cell metaplasia, fibrogenesis, red blood cell dysfunction and coagulation, and increased cellular senescence of epithelial and endothelial cells (D′Agnillo et al., 2021; Laforge et al., 2020). To overcome this ineffective tissue repair, several studies have suggested mesenchymal stem cell therapy as a treatment for COVID-19 (Esquivel et al., 2021; Rajarshi et al., 2020). The cell proliferation and growth markers identified in this study may have roles in regulating, and dysregulating, the process of tissue repair in COVID-19.

Of these growth markers, mesothelia (MSLN) is particularly interesting. Mesothelin is a differentiation antigen that has been almost exclusively studied in the context of malignant cancers such as mesothelioma, renal carcinoma, pancreatic cancer, ovarian cancer, and lung adenocarcinoma (Pastan & Hassan, 2014; Hassan et al., 2004), and was one of the most significantly elevated proteins in our dataset. While it is an attractive target for cancer immunotherapies due to its overexpression on cancer cells versus low expression on normal human tissue (Hassan & Ho, 2008), its function under normal physiological conditions is still largely unknown. It has been shown to bind mucin 16 (MUC16, aka. CA125) (Kaneko et al., 2009), and this relationship has been suggested to contribute to metastasis (Muniyan et al., 2016); however, mouse models have shown it is not required for normal development or reproduction (Bern & Pastan, 2000), and its biological function remains ambiguous. More research is required to not only identify the mechanism by which mesothelin augments COVID-19 pathogenesis, but also the biological role of mesothelin in the lung.

Also particularly interesting are a subset of proteins significantly elevated in milder COVID-19 disease versus severe (TRANCE, FASLG, XPNPEP2, and CD207), these represent an “efficient” disease response and improved prognoses. TRANCE and FASLG were also significantly elevated in response to SARS-CoV-2 infection in subjects 18-35 years old compared to those over 65 years old. These two proteins are already known to have critical roles in regulation of the T-cell response to viral infection, and activation of the signaling pathways associated with these proteins would indicate that a robust induction of T-cell activity has taken place in response to viral infection. As mentioned above, TRANCE and FASLG are both markers T-cell activity and it is established that T-cell exhaustion is associated with more severe COVID-19 disease (Song et al., 2020a). TRANCE is upregulated in T-cells following antigen receptor stimulation, and promotes dendritic cell-mediated stimulation of naïve T-cells (Leibbrandt & Penninger, 2008); furthermore, mutations in TRANCE have been found to associate with higher chronicity of other viral infections (Huang et al., 2021). While upregulation has previously been reported to be associated with severe COVID-19, and chronically elevated FASLG is known to increase with aging (Leopardi et al., 2022; de Almeida Chuffa et al., 2022), none of these studies have directly compared circulating protein levels in severe cases to mild and moderate disease and chronic elevation may correlate to an inadequate T-cell response to viral infection in older people. Additionally, while these studies suggest that elevated expression of FASLG is indicative of its role in T-cell- and NK-cell-mediated apoptosis, FASLG also functions as a modulator of T-cell differentiation through non-apoptotic signaling, and facilitates the clearance of activated T-cells and B-cells cells (Nagata, 1999) as well as promoting the resolution of type 2 lung inflammation (Williams et al., 2018; Sharma et al., 2012). Therefore, a multifaceted role for elevated FASLG expression in COVID-19 is not necessarily contradictory, and this pathway has been previously proposed as a mechanism behind the abnormal T-cell activity and subsequent exhaustion observed in severe cases of the disease (Leonardi et al., 2022; Leonardi & Proenca, 2020). Additionally, other published studies have either only focused on mRNA concentrations and not protein (Ramljak et al., 2021), or compared “severe” ICU patients to “moderate” non-ICU hospitalized patients (Andre et al., 2022), and not to “mild” cases. The lower levels observed in severe and moderate cohorts in this study may reflect the conclusion of the process of T-cell exhaustion, while the mild cohort maintains T-cell activation and proliferation at the time of sample collection. The data suggests that a significantly higher activation of FASLG in response to infection occurs in milder disease. Therefore, the significantly augmented levels of FASLG protein associated with severity and age suggest that circulating levels of these proteins may correlate to a more effective response to SARS-CoV-2 infection. Our data underlines the potential importance of these proteins as targets for stimulating an effective response to SARS-CoV-2 infection and require further investigation in this context.

XPNPEP2, a bradykinin-degrading hydrolase, is heavily associated with ACE activity (Rasmussen et al., 2020), and variants in the coding gene are associated with higher risk for ACE-inhibitor induced angioedema (Pall et al., 2021; Montinaro & Cicardi, 2020). Given that a “bradykinin storm” has been implicated in elevating disease severity during SARS-CoV-2 infection (Garvin et al., 2020; van de Veerdonk, et al., 2020; Roshe & Roche, 2020), and given XPNEP2's function in degrading bradykinin, elevated XPNPEP2 expression may have a potential protective effect in decreasing the inflammatory effects of bradykinin signaling, an effect supported by the data presented in this study. Due to its association with ACE, XPNPEP2 has been previously identified as potentially involved in SARS-CoV-2 infection in exploratory analyses using protein-protein interaction network databases (Khodadoost et al., 2020; Saih et al., 2021), and has been significantly associated with asymptomatic COVID-19 versus both symptomatic COVID-19 and healthy controls in pregnant women (Foo et al., 2021). However, XPNPEP2 has not yet been thoroughly investigated as a modulator of COVID-19 disease severity.

CD207, also known as langerin, is involved in efficient antigen presentation in DCs (Idoyaga, et al., 2008) and has been shown to bind the SARS-CoV-2 spike protein (Gu et al., 2022), suggesting that it may play a role in viral entry, though this role has not been investigated further. However, studies showing that impairment of DC numbers and function is associated with severe COVID-19, and that this impairment can persist long after resolution of infection (Zhou et al., 2020; Perez-Gomez et al., 2021), along with observation of an increase in mature DCs in the bronchoalveolar lavage fluid of COVID-19 patients versus healthy controls (Xiong et al., 2020), support an important role for DCs in mediating COVID-19 disease response. In addition, our data supports further investigation into CD207's interactions with SARS-CoV-2 and its role in efficient DC-mediated disease response.

In conclusion, analysis of protein concentration in plasma samples of patients infected with SARS-CoV-2 and presenting with differential severity of disease has identified proteins as biomarkers of both severe and effective responses to viral infection in mild cases. These findings simultaneously assess a broad range of physiological responses to the disease, finding significant markers associated with multiple inflammatory pathways, viral entry and membrane fusion, endothelial damage, and cell proliferation and tissue repair, highlighting the complexity of COVID-19 pathogenesis. These proteins and associated signaling pathways are potential targets for therapeutic intervention to both stimulate an effective response to SARS-CoV-2 infection or to prevent progression to severe disease.

### Example II

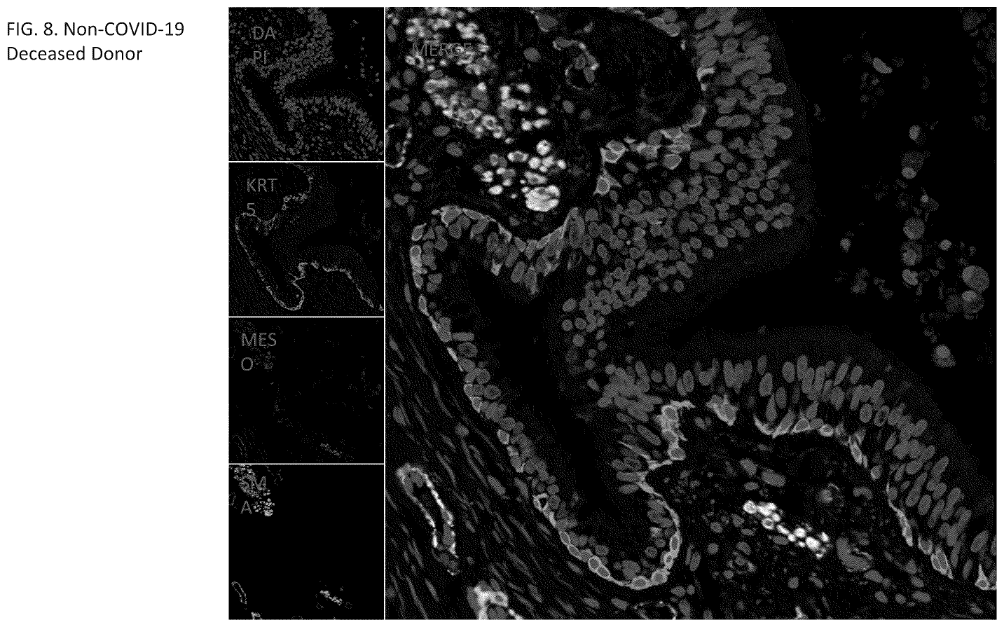

There was significantly higher expression of mesothelin in Severe COVID-19 lung tissues compared to controls (see FIGS. 7-8). There was also significant transcript expression in patient airway cells. Typically, mesothelin is expressed at low levels in the lung so this supports a pathological role in severe COVID-19 disease and a biomarker of severe disease,

### Example III

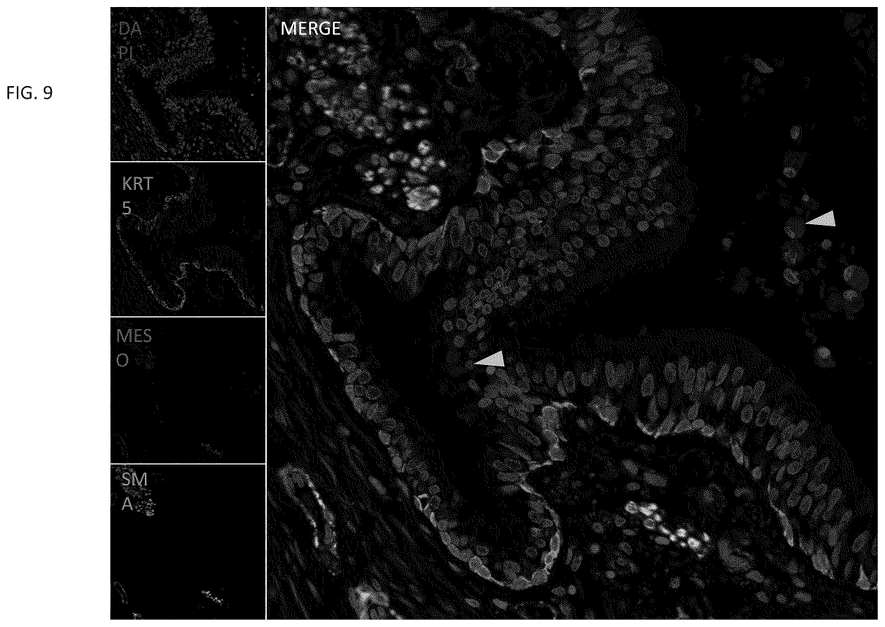

As lung disease is the is the prominent cause of morbidity and mortality in COVID-19 the expression of Mesothelin in lung tissue was investigated by comparing the airways of Non-COVID-19 subject donor tissues with tissues from patients who passed from COVID-19 (severe disease). Staining of lung tissues is shown in FIGS. 9-12 where a significant increase in epithelial Mesothelin (red) can be observed in the COVID-19 tissues compared to the Non-COVID-19. This data supports plasma levels of circulating Mesothelin and suggests that circulating Mesothelin expression may correlate with an increased Mesothelin expression in severe COVID-19 subject lung tissue.

For example, in FIG. 9, low amounts of mesothelin were associated with the epithelial cell layer where the basolateral surface was marked by green keratin 5 labelled basal cells. This expression is observed in a region of epithelial damage.

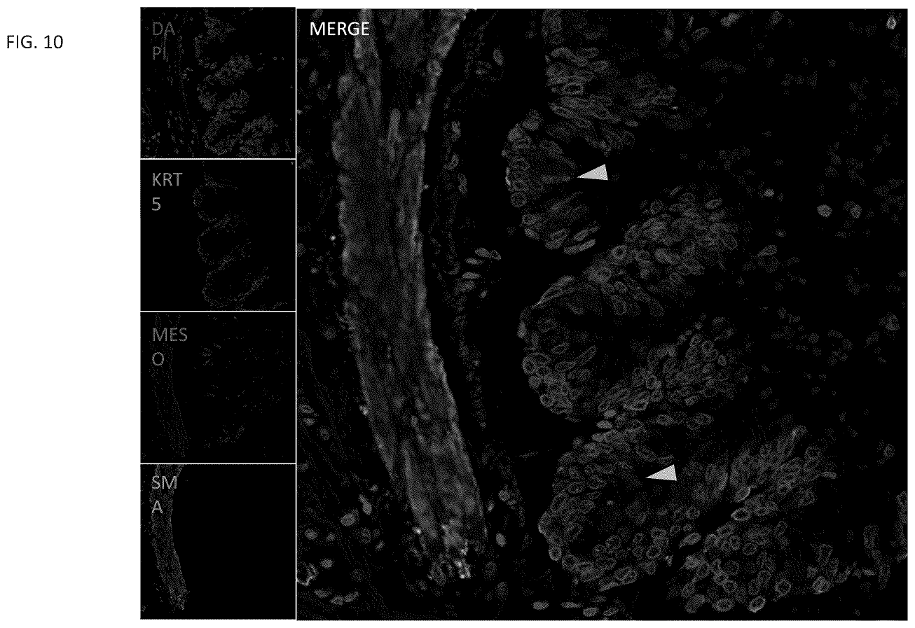

FIG. 10 shows that in COVID-19 lung tissue, there is a substantial increase in Mesothelin expression in the luminal cells of the epithelium. These comprise secretory cells, ciliated cells and ionocytes.

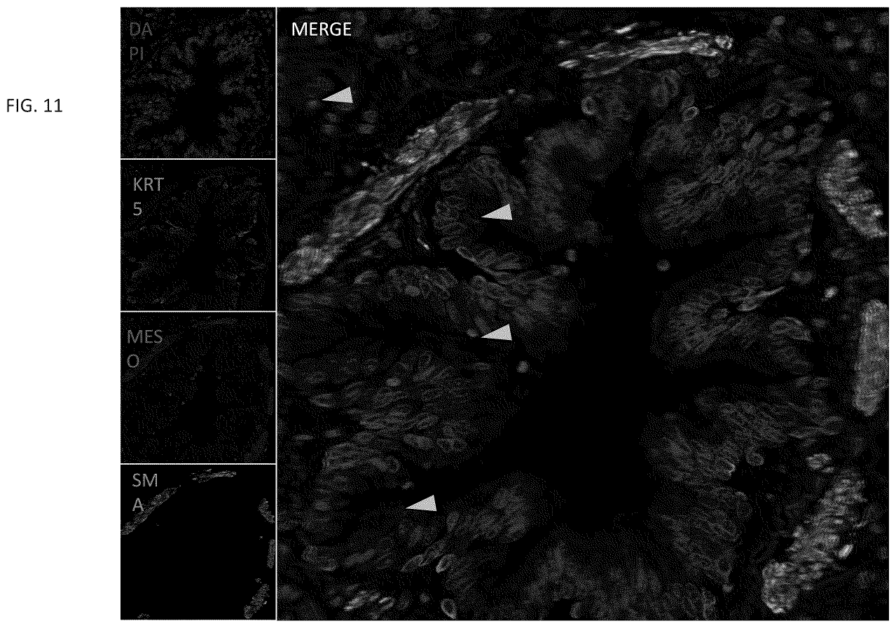

In COVID-19 lung tissue there is a substantial increase in Mesothelin expression also associated with smooth muscle actin expressing cells (FIG. 11). These comprise secretory cells, ciliated cells and ionocytes.

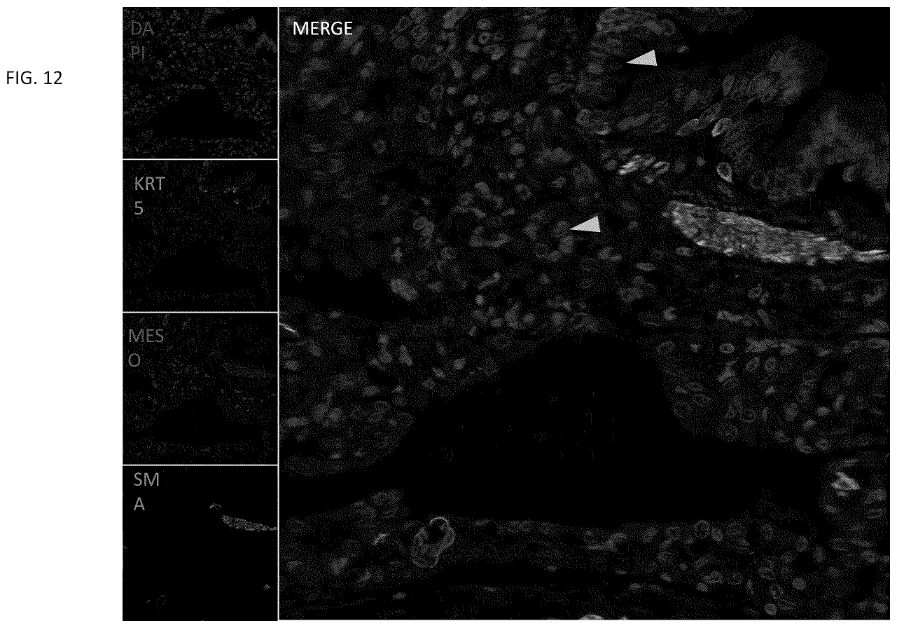

Increased mesothelin is observed in lung tissue where immune cell infiltration is observed (FIG. 12).

Single-cell RNA data from CZ CELLxGENE Discover was accessed, queried and analyzed and Mesothelin compared over two available datasets from studies evaluating COVID-19 and non-COVID-19 subjects. See

DOI: https://doi.org/10.1038/s41591-023-02327-2

Celll X gene: https://cellxgene.cziscience.com/e/9f222629-9e39-47d0-b83f-e08d610c7479.cxg/

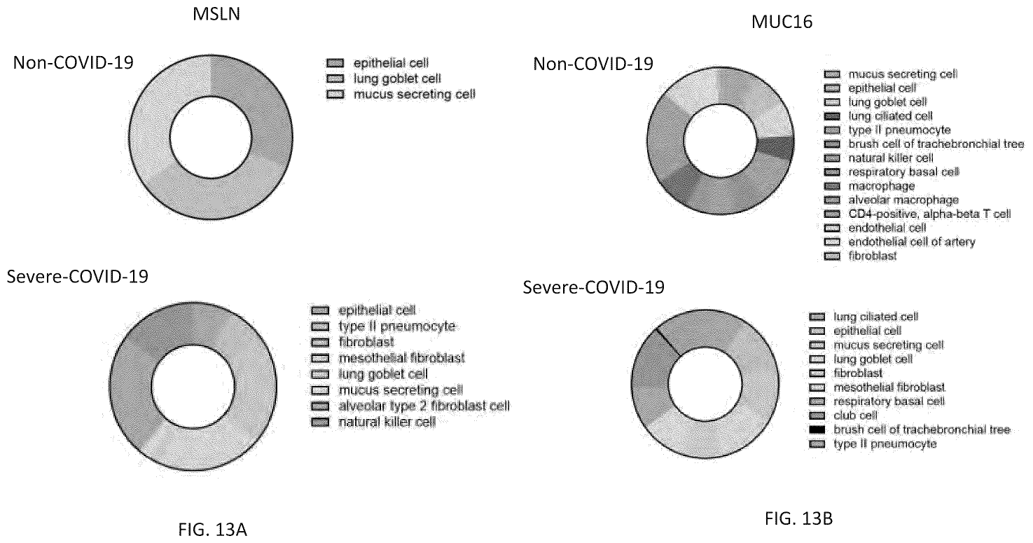

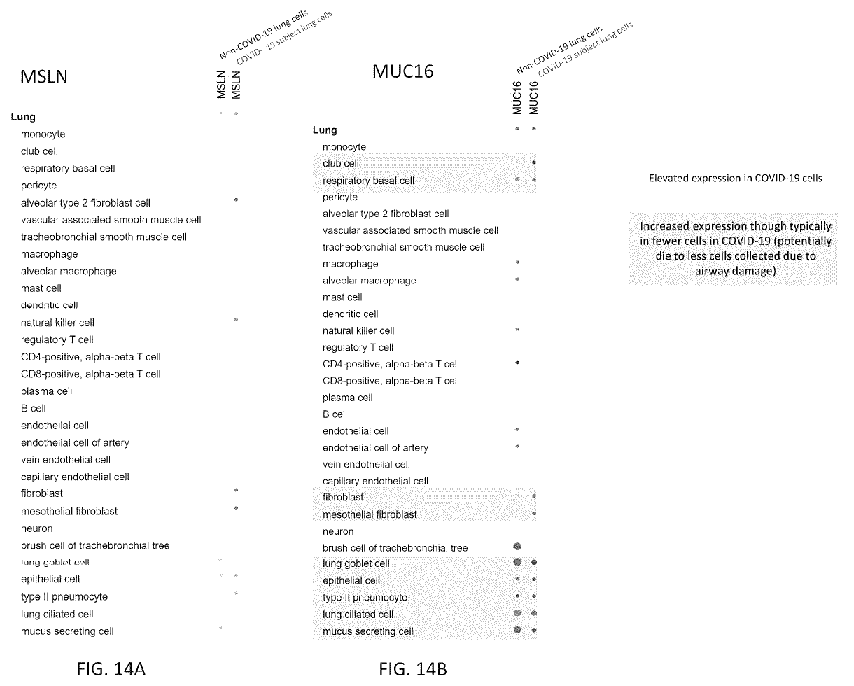

While transcript levels of the MSLN gene were not notably elevated, it was clear that MSLN was expressed in a much wider variety of airway cells in COVID-19, as shown in the plots in FIG. 13.. In addition, the cell types in the lung expressing MUC16, a known receptor for secreted MSLN, were decreased while the transcript levels in those cells expressing it increased (FIG. 14) (doi: 10.1038/srep01870).

### References

Abrehart et al., Paediatr. Respir. Rev., : (2022).
Acevedo et al., Front. Immunol., 12:678661 (2021).
Andre et al., Cell Death Differ., : (2022).
Bera & Pastan, Mol. Cell Biol., 20:2902 (2000).
Bermejo-Jambrina et al., EMBO J., 40:e106765 (2021).
Blank et al., Nat. Rev. Immunol,,: 19:665 (2019).
Bosmuller et al., Virchows Arch., 478:137 (2021).
Bragado et al., Oncotarget., 11:4541 (2020).
Buttenschon et al., H N, Lynggaard V, Sandbol S G, Glassou E N, and Haagerup A. Comparison of the clinical 370 presentation across two waves of COVID-19: a retrospective cohort study. BMC Infect. Dis., 22:423 (2022).
Cannarile et al., J. Immunother. Cancer. 5:53 (2017).
Chen et al., bioRxiv, : (2022).
Chen et al., Br. J. Cancer, 126:754 (2022).
Chiappalupi et al., Biomolecules. 11: (2021b).
Chiappalupi et al., Life Sci., 272:119251 (2021a).
Chiumento et al., Influenza Other Respir. Viruses, 16:532 (2022)
Cohen et al., Nat. Commun., 12:4678 (2021).
D'Agnillo et al., Sci. Transl. Med., 13:eabj7790 (2021).
de Almeida Chuffa et al., J. Mol. Med. (Berl)., 100:285 (2022).
de Miranda et al., Trans. R. Soc. Trop. Med. Hyg., 362: (2022)
Desai et al., Am. J. Physiol. Cell Physiol., 322:C1 (2022).
Dupont et al., Arterioscler. Thromb. Vasc. Biol., 41:1760 (2021).
Esquivel et al., Stem Cell Rev. Rep., 17:144 (2021).
Feng et at., Cancer Res., 80:4386 (2020).
Ferrario et al., Circulation, 111:2605 (2005). 385
Files et al., J. Clin. Invest., 131: (2021).
Foell et al., Lancet, 361:1270 (2003c).
Foell et al., Rheumatology (Oxford), 42:1383 (2003a).
Foell et al., Thorax, 58:613 (2003b).
Foo et al., Cell. Rep. Med., 2:100453 (2021).
Garvin et al., Elife, 9: (2020).
Gerayeli et al., EClinicalMedicine, 33:100789 (2021).
Gu et al., Cell. Res., 32:24 (2022).
Goan et al., N. Engl. J. Med. 382:1708 (2020).
Gubbels et al., Mol. Cancer, 5:50 (2006).
Hassan & Flo, Eur. J. Cancer, 44:46 (2008).
Hassan et al., Clin. Cancer Res., 10:3937 (2004).
Higgins et al., Proc. Natl. Acad. Sci. USA, 111:10648 (2014).
Hoffmann et al., Cell, 181:271 e8 (2020).
Flu et al., J. Med. Virol., 93:250 (2021).
Huang et al., Front Med. (Lausanne), (2021)8:743406.
Hudaket al., Int. J. Mol. Sci., 22: (2021).
Idoyaga et al., J. Immunol., 180:3647 (2008).
Iulita R. Cuello, Curr. Alzheimer Res., 13:53 (2016).
Johansson et al., Ann. Snug., 254:194 (2011).
Karampoor et al., Int. Immunopharmacol., 97:107684 (2021).
Khodadoost et al., Gastroenterol. Hepatol. Bed. Bench., 13:374 (2020).
Laforge et al., Nat. Rev. Immunol., 20:515 (2020).
Leibbrandt & Penninger, Ann. N.Y. Acad. Sci., 1143:123 (2008).
Leonardi & Proenca, Front. Immunol., 11:600405 (2020).
Leonardi et al., Front. Immunol., 13:853606 (2022).
Letko et al., Nat. Microbiol., 5:562 (2020).
Li et al., Onco. Targets Ther., 11:731 (2018).
Li et al., Oncotarget., 7:81026 (2016).
Ligthart et al., Arterioscler. Thromb. Vasc, Biol., 34:2695 (2014).
Loske et al., J, Rohmel J, Lukassen S, Stricker S, Magalhaes V G, Liebig J, et al., Nat. Biotechnol., 40:319 (2022).
Luo et al., Scand. J. Immunol., 93:e12989 (2021).
Ma et al., J. Cell. Mol. Med., 25:7001 (2021).
Maldonado et al., Front Med. (Lausanne), 9:826218 (2022).
Mikami et al., J. Gen. Intern. Med., 36:17 (2021).
Montinaro & Cicardi, Int. Immunopharmacol., 78:106081 (2020).
Muniyan et al., Genes Cancer, 7:110 (2016).
Nagata, Annu. Rev. Genet., 33:29 (1999).
Ogawa et al., Thromb. J., 19:55 (2021).
Ostergaard, Physiol. Rep., 9:e14726 (2021).
Pall et al., Pharmacogenomics, 22:319 (2021).
Palmer et al., J. R. Soc. Interface, 18:20200982 (2021)
Pastan & Hassan, Cancer Res., 74:2907 (2014),
Perez-Gomez et al., Cell. Mol. Immunol. 18:2128 (2021).
Petrara et al., Front. Immunol., 12:741796 (2021).
Pierce et al., JCI Insight., 6: (2021).
Rajarshi et al., Biotechnol. Rep. (Amst)., 26:e00467 (2020).
Ramljak et al., J. Pers. Med., 11: (2021).
Rasmussen et al., Pharmacogenornics J., 20:770 (2020).
Rass et al, Eur. J. Neurol., 29:1685 (2022).
Renelus et at., J. Racial. Ethn. Health Disparities. 8:1161 (2021)
Roche & Roche, FASEB J., 34:7265 (2020).
Russell et al., Am. J. Respir. Cell. Mol. Biol., 66:196 (2022).
Saih et al., Biomed. Res. Int., 2021:6685840 (2021).
Schafer et al., Sci. Rep., 12:7249 (2022).
Schultze & Aschenbrenner, Cell, 184:1671 (2021).
Sharma et al., Allergy, 67:328 (2012).
Singh et al., Cell Rep., 32:108175 (2020). 379
Song et al., Clin. Chim. Acta, 509:280 (2020b).
Song et al., Curr. Med. Sci., 41:14 (2021).
Song et al., Nat. Commun., 11:3410 (2020a).
Stahl et al., Am, J. Respir, Crit. Care Med., 202:1178 (2020).
Suleyman et al., Open Forum Infect. Dis., 9:ofac116 (2022).
Teng et al., Matrix Biol., 31:3 (2012).
Thwaites et al., Sci. Immunol., 6: (2021).
Tserel et at., Front. Immunol., 12:709759 (2021).
Ushach & Zlotnik, J. Leukoc. Biol., 100:481 (2016).
van de Veerdonk et al., Elife, 9: (2020).
Varga et al., Lancet, 395:1417 (2020).
von Mering et al., Nucleic Acids Res., 33:D433 (2005).
Wang et al., J. Leukoc. Biol., 108:17 (2020).
Wang et at., JAMA, 323:1061 (2020).
Westmeier et al., mBio. 11: (2020).
Williams et al., Front. Immunol., 9:2521 (2018),
Wu &. McGoogan, JAMA, 323:1239 (2020).
Wu et al., J. Transl. Med., 18:384 (2020).
Xiong et al., Emerg. Microbes Infect., 9:761 (2020).
Yang et at., Infect. Dis. Poverty, 9:161 (2020).
Yoshimatsu et al., PLoS One, 15:e0232356 (2020).
Zeng et al., FEBS J., 288:5190 (2021).
Zhang et al., Mol. Med., 27:151 (2021).
Zhou et al., Immunity. 53:864 (2020).
Zhu et al., N. Engl. Med., 382:727 (2020).
Zimmermann & Curtis, Pediatr. Infect. Dis. J., 41:e36 (2022).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to determine an increased risk of developing severe COVID in a mammal, comprising:
determining an amount of mesothelin in plasma or serum from a test mammal infected or suspected of being infected with SARS-CoV-2; and
determining whether the amount of mesothelin in the plasma or serum of the test mammal is greater than a corresponding sample from a corresponding mammal that does not have severe COVID, wherein if the amount in the test mammal is greater than the mammal that does not have severe COVID, the test mammal is at increased risk of developing severe COVID.

2. The method of claim 1 wherein the mammal that does not have severe COVID has mild or moderate COVID.

3-4. (canceled)

5. The method of claim 1 wherein the mammal is a human.

6. The method of claim 1 further comprising treating the test mammal having greater amounts of mesothelin in the plasma or serum with one or more compounds.

7. The method of claim 6 wherein the one or more compounds include an anti-viral compound or an anti-mesothelin compound, or a combination thereof, optionally wherein the one or more anti-viral compounds include an antibody or fragment thereof a protease inhibitor, a steroid, a ribonucleoside or a ribonucleotide.

8-14. (canceled)

15. The method of claim 7 wherein the one or more anti-mesothelin compounds comprise CAR-T therapy.

16. The method of claim 6 wherein the one or more compounds include an IL-6 inhibitor or a JAK inhibitor.

17. (canceled)

18. A method to prevent, inhibit or treat SARS-CoV-2 infection in a mammal, comprising administering an effective amount of one or more compounds to a mammal determined to have increased mesothelin levels in plasma or serum relative to a corresponding sample from a corresponding mammal that is infected with SARS-CoV-2 and does not have severe COVID or a corresponding sample in a corresponding mammal that is not infected with SARS-CoV-2.

19. The method of claim 18 wherein the corresponding mammal has moderate or mild COVID.

20. (canceled)

21. The method of claim 18 wherein the mammal is a human.

22. The method of claim 18 wherein the one or more compounds include an anti-viral compound or an anti-mesothelin compound, or a combination thereof.

23. The method of claim 22 wherein the one or more anti-viral compounds include an antibody or fragment thereof.

24. The method of claim 23 wherein the antibody is a monoclonal antibody or a fragment thereof.

25. The method of claim 22 wherein the one or more anti-viral compounds include a protease inhibitor, a ribonucleoside or a ribonucleotide.

26. (canceled)

27. The method of claim 18 wherein the one or more compounds include a steroid.

28. The method of claim 18 wherein the one or more anti-mesothelia compounds comprise Car-T therapy.

29. The method of claim 18 wherein the one or more compounds include an IL-6 inhibitor.

30. The method of claim 18 wherein the one or more compounds include a JAK inhibitor.

31. A method comprising:
determining an amount of mesothelin in plasma or serum from a mammal or a patient having COVID; and
determining whether the amount of mesothelin in plasma or serum in the mammal or the patient is greater than a corresponding sample from a corresponding mammal or patient that does not have severe COVID, wherein if the amount in the mammal having COVID is greater than the corresponding mammal that does not have severe COVID, the mammal is at an increased risk of developing long COVID or wherein if the amount in the patient with COVID is greater than the patient that does not have severe COVID, the patient is a candidate for admission to the ICU.

32-33. (canceled)

34. A method to prevent, inhibit or treat SARS-CoV-2 infection in a mammal, comprising administering to a mammal having increased mesothelin levels in plasma or serum relative to a corresponding sample from a corresponding mammal that is infected with SARS-CoV-2 and does not have severe COVID, an amount of one or more compounds that bind to or mesothelin.
